(19) United States
(12) Patent Application Publication
(10) Pub. No.: US 2023/0084515 A1
(43) Pub. Date: Mar. 16, 2023

(54) THERAPEUTIC METHODS AND COMPOSITIONS FOR TREATING CANCER USING BRAF AND/OR MEK INHIBITOR COMBINATION THERAPY

(71) Applicants: Vanderbilt University, Nashville, TN (US); Duet BioSystems, Inc., Nashville, TN (US)

(72) Inventors: Laurent Audoly, Brookline, MA (US); Buddhi Bishal Paudel, Nashville, TN (US); Vito Quaranta, Nashville, TN (US)

(21) Appl. No.: 17/800,394

(22) PCT Filed: Feb. 19, 2021
(86) PCT No.: PCT/US21/18822

§ 371 Date: Aug. 17, 2022

Related U.S. Application Data
(60) Provisional application No. 62/978,538, filed on Feb. 19, 2020.

(51) Int. Cl. A61K 31/437; A61K 31/506; A61K 31/4439; A61K 31/4184; A61K 31/4523; A61K 31/352; A61K 31/519; A61K 31/11; A61K 31/33; A61K 31/635; A61K 31/517; A61K 31/198; A61P 35/00

(52) U.S. Cl. CPC A61K 31/437; A61K 31/506; A61K 31/4439; A61K 31/4184; A61K 31/4523; A61K 31/352; A61K 31/519; A61K 31/11; A61K 31/33; A61K 31/635; A61K 31/517; A61K 31/198; A61P 35/00

(57) ABSTRACT

The invention provides methods, compositions, and medical kits for treating cancer using combination therapy including a BRAF and/or MEK inhibitor and at least one additional therapeutic agent that inhibits at least one target impacting oxidation state of the cancer.

### RELATED APPLICATIONS

This application claims the benefit of U.S. Provisional Patent Application No. 62/975,538, filed Feb. 19, 2020, entitled “THERAPEUTIC METHODS AND COMPOSITIONS FOR TREATING CANCER USING BRAF AND/OR MEK INHIBITOR COMBINATION THERAPY,” the entire contents of which are incorporated herein by reference.

### STATEMENT REGARDING FEDERALLY SPONSORED RESEARCH

This invention was made with government support under contracts U54-CA217450 and U54-CA113007 both awarded by the National Institutes of Health. The government has certain rights in the invention.

### FIELD OF THE INVENTION

The invention provides methods, compositions, and medical kits for treating cancer using a combination therapy including a BRAF and/or MEK inhibitor and at least one additional therapeutic agent that inhibits a target impacting oxidation state of the cancer.

### BACKGROUND

Cancer is a leading cause of death in many industrialized countries. Recent estimates are that 10 million Americans are currently living with cancer, and that 1.2 million Americans are newly diagnosed with cancer each year. Significant advances have been made in improving the diagnosis and treatment of cancer. However, current treatment options often suffer from severe adverse side effects and/or the treatments are not effective for all patients. For example, many clinically-accepted chemotherapeutic agents can induce profound damage to normal, proliferative host cells. Another problem associated with many chemotherapeutic treatments is that, in many tumor types, there is either inherent or acquired resistance to the therapy.

Approximately 200,000 individuals per year are newly diagnosed with melanoma worldwide. Melanoma is characterized by the formation of malignant melanocytes. Exemplary therapies currently used to help treat patients suffering from prostate cancer include surgery, radiation therapy, immunotherapy, and chemotherapy. Surgery is often effective for localized melanoma, but is often insufficient for treating metastatic melanoma. Radiation therapy involves applying ionizing radiation to the diseased area of the prostate. However, not all patients achieve sufficient remission of melanoma, especially metastatic melanoma, using existing therapies and/or experience adverse side effects when subjected to existing therapies.

Accordingly, the need exists for new therapeutic methods that provide improved efficacy and/or reduced side effects for treating melanoma. The present invention addresses this need and provides other related advantages.

### SUMMARY

The invention provides methods, compositions, and medical kits for treating cancer using a combination therapy including a BRAF and/or MEK inhibitor and at least one additional therapeutic agent that inhibits a target impacting oxidation state of the cancer. The cancer may be, for example, a melanoma. The BRAF and/or MEK inhibitor is used in combination with a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, U3, ERBB4, IP3, MCU, SLC16A7, ELF3, NROB1 or EPHA2. The methods, compositions, and medical kits for treating cancer of the present disclosure provide particular benefits to patients suffering from cancers having a BRAF mutation and/or a KRAS mutation.

Accordingly, one aspect of the invention provides a method for treating cancer in a patient. The method comprises administering to a patient in need thereof a therapeutically effective amount of a first therapeutic agent and a second therapeutic agent to treat the cancer, wherein the first therapeutic agent comprises a BRAF and/or MEK inhibitor, and the second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2. In certain embodiments, the cancer is a melanoma. The method may be characterized according to, for example, the type of melanoma, such as superficial spreading melanoma, nodular melanoma, acral-lentiginous melanoma, lentigo maligna melanoma, amelanotic melanoma, desmoplastic melanoma, ocular melanoma, nevoid melanoma, or spitzoid melanoma. In certain embodiments, the melanoma is metastatic melanoma. Further embodiments are described herein below.

Another aspect of the invention provides a method for selecting a patient for treatment of cancer, the method comprising analyzing a sample from the patient to identify at least one of:
a. elevated expression levels of at least one target;
b. elevated functional activity of at least one target; or
c. elevated oxidation state of the cancer;
   wherein the target is independently selected from the group consisting of NOX5, SLC7A11, GSH, GPX, CYBA, EDG2, PPP1CC, PP1C, PP1gamma, ROCK2, RPS6KA2, SYK-AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 and EPHA2;
      wherein upon identification of one or more of (a) elevated expression levels of at least one target, (b) elevated functional activity of at least one target, or (c) elevated oxidation state of the cancer, the patient is selected for treatment using a first therapeutic agent and a second therapeutic agent to treat the cancer, wherein the first therapeutic agent comprises a BRAF inhibitor and/or a MEK inhibitor, and the second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2. In certain embodiments, the cancer is melanoma. In certain embodiments, the target is NOX5. Further embodiments are described herein below.

Another aspect of the invention provides a medical kit for treating a cancer. The kit comprises (i) a first therapeutic agent comprising a BRAF and/or MEK inhibitor; (ii) a second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, SLC16A7, ELF3, NROB1 or EPHA2; and (iii) instructions for treating a cancer using said first therapeutic agent and said second therapeutic agent. The instructions may specify, for example, the route of administration for the first therapeutic agent and the second therapeutic agent, such as by oral, intravenous, or parenteral administration. In certain embodiments, the cancer is melanoma. In certain embodiments, the target is NOX5. Further embodiments are described herein below.

The foregoing aspects of the invention are described in more detail, along with additional embodiments, in the detailed description below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict the identification of molecular determinants of short-term drug response variability using single-cell derived isogenic sublines. FIG. 1A contains data from previous reports showing drug-response variation among BRAF-mutated melanoma cells at the population level and among single-cell derived sublines of SKMEL5 cell line at the clonal level. For both population and clonal level, drug response is quantified as DIP rates (doublings/h) for cellular response at 8 μM PLX4720. FIG. 1B is a heat map of differentially expressed genes determined after pairwise comparisons between SC07 vs SC01, SC10 vs SC01 and SC10 vs SC07. Three SKMEL5 sublines: SC01, SC07 and SC10 were chosen for RNASeq analysis based on their divergent drug responses.

FIGS. 2A, 2B and 2C depict RNASeq analyses identifying NOX5 and PGC1α as potential molecular determinants of short-term response variability. (FIG. 2A) Number of differentially expressed genes (DEGS) from pairwise comparisons between sublines; red represents upregulated, blue represents downregulated genes. (FIG. 2B) Top KEGG pathway enriched in common sets of upregulated genes among sublines. −log₁₀ of adjusted p-valued is plotted along x-axis. (FIG. 2C) Volcano plots of differentially expressed genes from pairwise comparisons between sublines. Blue genes represent differentially expressed genes with FDR <0.001 and log fold change of 3 or higher. Indicated in magenta is NOX5 and indicated in red is PGC1α. log₂ of fold change in expression is plotted along x-axis and −log₁₀ of adjusted p-value is plotted along y-axis.

FIGS. 3A, 3B, 3C, and 3D depict graphs showing the correlation of combined expression of NOX5 and PGC1α with BRAFi DIP rates. (FIG. 3A) (left) Expression of NOX5 in sublines relative to its expression in SC01; (right) Correlation of NOX5 expression and DIP rates in SKMEL5 sublines. (FIG. 3B) (left) Expression of PGC1α in sublines relative to its expression in SC01; (right) Correlation of PGC1α expression and DIP rates in SKMEL5 subtitles. (FIG. 3C) (left) Combined expression of NOX5 and PGC1α in sublines relative to SC01; (right) Correlation of combined NOX5 and PGC1α expression and DIP rates in SKMEL5 sublines. In each of FIGS. 3A, 3B and 3C, reported are the correlation coefficients from Pearson Correlation. Sublines are placed in order of their increasing DIP rates, from left to right. (FIG. 3D) Sublines are placed in 2D landscape based on expression of NOX5 and PGC1α. All expressions reported are relative to SC01.

FIGS. 3E and 3F depict graphs showing the correlation of NOX5 expression with drug response in BRAF-mutated melanoma cells (FIG. 3E) NOX5 expressions obtained from Cancer Cell Line Encyclopedia (CCLE) correlated with Drug-Induced Proliferation Rates (DIP) rates of the respective BRAF-mutated melanoma cell lines at 8 uM PLX4720 (BRAFi). Pearson correlation was used to calculate the correlation between expression and drug response. Corr: 0.649, p-value=0.042. (FIG. 3F) NOX5 expression of different BRAF-mutated melanoma cell lines quantified and validated by quantitative PCR (qPCR), normalized to the expression of NOX5 in SC01. Pearson correlation was used to calculate the correlation between the normalized expression and the respective drug response, DIP rates at 8 uM PLX4720, Corr: 0.815, p-value=0.002

FIGS. 4A and 4B depict graphs showing that NOX5 mRNA expression is higher in skin tumor compared to normal skin but not PGC1α expression. (FIG. 4A) NOX5 mRNA expression levels were assessed in normal and cancer tissues of skin, (FIG. 4B) PGC1α mRNA expression levels were assessed in normal and cancer tissues of skin; both expression data retrieved from the Gene Expression Database of Normal and Tumor Tissues (GENT) database. Both expressions were significant, p<0.001

FIGS. 5A, 5B and 5C depict results showing the synergism between NADPH Oxidase (NOX) and BRAF-inhibition. (FIG. 5A) Heat map of DIP rates in various concentrations of DPI (NOXi) and PLX4720 (BRAFi). Black solid line separates drug-effects with positive DIP rates (top left) from negative DIP rates (bottom right). (FIG. 5B) (left) Population growth curves (log2 normalized) for SC10 in three NOXi (DPI, Apocynin, and GKT137831) and BRAFi (PLX4720) and their combination; (right) DIP rates quantified from population growth curves shown for DMSO control, BRAFi, three NOXi and their combination with BRAFi. (FIG. 5C) (left) qPCR Quantification of NOX5 transcript expression in siRNA-mediated NOX5 knockdown compared to Scrambled control. (FIG. 5C) (right) Population growth curves (log2 normalized) for SC10 in either DMSO, Scrambled control, siRNA, BRAFi and BRAFi+siRNA, Quantified. DIP rates in corresponding conditions. In both FIG. 5B and FIG. 5C, concentration of PLX4720 used was 8 μM.

FIGS. 6A and 6B depict graphs showing the enhancement of NOX-inhibition on the anti-proliferative effects of BRAF-inhibition. (FIG. 6A) (top) Population growth curves (log₂ normalized) for A2058 in either DPI (250 nM) or BRAFi (8 μM), or combination; (bottom) DIP rates quantified for corresponding conditions for respective growth curves. (FIG. 6B) (top) Population growth curves (log, normalized) for A2058 in either DMSO, Scrambled control, siRNA, BRAFi and BRAFi+siRNA; (bottom) Quantified absolute DIP rates or normalized DIP rates (normalized to the control in respective controls) in corresponding conditions, * denotes p<0.05 and *** denotes p<0.001.

FIG. 7 is a graph showing the correlation of NOX5 expression with treatment outcome. Gene expression values from drug-naive patient samples were obtained from three published reports and quantile normalized before comparison. Samples were placed in either Responders or Non-Responders category based on RECIST criteria. Responders included partial and complete response (PR, CR); and Non-Responders included stable and progressive disease (SD, PD). Two-sided t-test was performed for statistical comparison.

FIGS. 8A and 8B depict graphs showing that NOX5 and PG-C1α expression define metabolic states of melanoma cells. (FIG. 8A) Correlation between expression of NOX5 in BRAF-mutated melanoma cells including isogenic sublines relative to SC01 and glycolytic reserve, indicative of glycolytic functions quantified from Seahorse assays. Pearson correlation coefficient: 0.776, p-value <0.05. (FIG. 8B) Correlation between expression of PGC1α in BRAF-mutated melanoma cells including isogenic sublines relative to SC01 and oxygen consumption rate, indicative of mitochondrial respiration quantified from Seahorse assays. Pearson correlation coefficient: 0.638, p-value not significant.

### DETAILED DESCRIPTION

The invention provides methods, compositions, and medical kits for treating cancer using a combination therapy including a BRAF and/or MEK inhibitor and at least one additional therapeutic agent that inhibits at least one target impacting oxidation state of the cancer. The cancer may be, for example, a melanoma. The BRAF and/or MEK inhibitor is used in combination with a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2. The methods, compositions, and medical kits for treating cancer provide particular benefits to patients suffering from cancers having a BRAF mutation and/or a KRAS mutation.

The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section.

### I. DEFINITIONS

To facilitate an understanding of the present invention, a number of terms and phrases are defined below.

The terms “a,” “an” and “the” as used herein mean “one or more” and include the plural unless the context is inappropriate.

The term “about” means within 10% of the stated value. In certain embodiments, value may be within 8%, 6%, 4%, 2%, or 1% of the stated value.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to organisms to be treated by the methods of the present invention. Such organisms preferably include, but are not limited to, mammals (e.g., murines, simians, equines (horses), bovines (cattle), porcines, canines, felines, and the like), and most preferably includes humans.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to any of the standard pharmaceutical carriers. The compositions also can include stabilizers and preservatives. For examples of carriers, stabilizers, and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, furnarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Still other examples of salts include anions of the compounds of the present invention compounded with a suitable cation such as Na⁺, NH₄⁺, and NW₄⁺ (wherein W is a C₁₋₄ alkyl group), and the like. The term “alkyl” is art-recognized, and includes saturated aliphatic groups, including straight-chain alkyl groups and branched-chain alkyl groups.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “therapeutically effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. THERAPEUTIC APPLICATIONS

One aspect of the invention provides a method of treating cancer in a patient. The method comprises administering to a patient in need thereof a therapeutically effective amount of a first therapeutic agent and a second therapeutic agent to treat the cancer, wherein the first therapeutic agent comprises a BRAF inhibitor and/or a MEK inhibitor, and the second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2.

### General Features of the Therapeutic Method

The above therapeutic method may be further characterized by additional features, such as the type of cancer treated, the patient's medical history, the identity of the first therapeutic agent(s), and the identity of the second therapeutic agent(s).

### Type of Cancer

The therapeutic method can be further characterized according to the type of cancer to be treated. For example, in certain embodiments, the cancer is a melanoma. In certain embodiments, the melanoma is a superficial spreading melanoma, nodular melanoma, acral-lentiginous melanoma, lentigo maligna melanoma, amelanotic melanoma, desmoplastic melanoma, ocular melanoma, nevoid melanoma, or spitzoid melanoma. In certain embodiments, the cancer is metastatic melanoma. In certain embodiments, the cancer is a melanoma that is not metastatic.

The cancer may be further characterized according to the presence of at least one mutation in the cancer. In certain embodiments, the cancer has a BRAF mutation. In certain embodiments, the BRAF mutation is a V600E or V600K mutation in the BRAF gene. In certain embodiments, the cancer has a KRAS mutation.

The method may be further characterized according to the prior treatment of the cancer. In certain embodiments, the patient has not previously received a BRAF inhibitor for treatment of the cancer. In certain embodiments, the cancer has been in remission for at least one month. In certain embodiments, the cancer has been in remission for at least two months. In certain embodiments, the cancer has been in remission for at least three months. In certain embodiments, the cancer has been in remission for at least six months.

The cancer may be further characterized according to whether it has elevated expression and/or activity of an enzyme or receptor. For example, in certain embodiments, the cancer is characterized by elevated expression levels of the target (i.e., elevated expression levels of one or more of NOX5, SLC7A11, GSH, GPX, CYBA, EDG2, PPP1CC, PP1C, PP1gamma, ROCK2, RPS6KA2, SYK-AKT1, AKT2, BP, GSR, G6PD, ABCB5, EPHA(i), DLK1, IDH1, ME2/3, HTATIP2, DKK1, RAC3, ERBB4, IP3, MCU, SLC16A7, ELF3, NROB1 or EPHA2). For example, in certain embodiments, the cancer expresses elevated levels of NOX5. In certain embodiments, the cancer expresses elevated levels of SLC7A11. In certain embodiments, the cancer expresses elevated levels of GSH. In certain embodiments, the cancer expresses elevated levels of GPX. In certain embodiments, the cancer expresses elevated levels of CYBA. In certain embodiments, the cancer expresses elevated levels of EDG2. In certain embodiments, the cancer expresses elevated levels of PPP1CC. In certain embodiments, the cancer expresses elevated levels of PP1C. In certain embodiments, the cancer expresses elevated levels of PP1gamma. In certain embodiments, the cancer expresses elevated levels of ROCK2. In certain embodiments, the cancer expresses elevated levels of RPS6KA2. In certain embodiments, the cancer expresses elevated levels of SYK. In certain embodiments, the cancer expresses elevated levels of AKT1. In certain embodiments, the cancer expresses elevated levels of AKT2. In certain embodiments, the cancer expresses elevated levels of BP. In certain embodiments, the cancer expresses elevated levels of GSR. In certain embodiments, the cancer expresses elevated levels of G6PD. In certain embodiments, the cancer expresses elevated levels of ABCB5. In certain embodiments, the cancer expresses elevated levels of EPHA(i). In certain embodiments, the cancer expresses elevated levels of DLK1. In certain embodiments, the cancer expresses elevated levels of IDH1. In certain embodiments, the cancer expresses elevated levels of ME2/3. In certain embodiments, the cancer expresses elevated levels of HTATIP2. In certain embodiments, the cancer expresses elevated levels of DKK1. In certain embodiments, the cancer expresses elevated levels of RAC3. In certain embodiments, the cancer expresses elevated levels of UQCRB. In certain embodiments, the cancer expresses elevated levels of ERBB4. In certain embodiments, the cancer expresses elevated levels of IP3. In certain embodiments, the cancer expresses elevated levels of MCU. In certain embodiments, the cancer expresses elevated levels of SLC16A7. In certain embodiments, the cancer expresses elevated levels of ELF3. In certain embodiments, the cancer expresses elevated levels of NROB1. In certain embodiments, the cancer expresses elevated levels of EPHA2.

Elevated expression levels of NOX5 may be identified by administering to the patient a PET ligand that binds to NOX5. The concentration of PET ligand bound to NOX5 in the cancer in the patient provides a measure of the level of expression of NOX5.

Elevated expression levels of SLC7A11 may be identified by administering to the patient a PET ligand that binds to SLC7A11. The concentration of PET ligand bound to SLC7A11 in the cancer in the patient provides a measure of the level of expression of SLC7A11.

In certain embodiments, the cancer expresses elevated levels of NOX5, SLC7A11, GSH, GPX, CYBA, EDG2, PPP1CC, PP1C, PP1gamma, ROCK2, RPS6KA2, SYK, BP, GSR, G6PD, ABCB5, EPHA(i), DLK1, ME2/3, HTATIP2, DKK1, RAC3, UQCRB, ERBB4, IP3,MCU, SLC16A7, ELF3, NROB1 or EPHA2.

In certain embodiments, the cancer expresses elevated levels of NOX5, SLC7A11, GSH, or GPX. In certain embodiments, the cancer expresses elevated levels of CYBA, EDG2, PPP1CC, PP1C, or PP1gamma. In certain embodiments, the cancer expresses elevated levels of ROCK2, EPHA(i), car EPHA2. In certain embodiments, the cancer expresses elevated levels of RPS6KA2 or SYK. In certain embodiments, the cancer expresses elevated levels of AKT1 or AKT2. In certain embodiments, the cancer expresses elevated levels of BP, GSR, G6PD, or ABCB5. In certain embodiments, the cancer expresses elevated levels of DLK1, IDH1, ME2/3, or HTATIP2. In certain embodiments, the cancer expresses elevated levels of DKK1, RAC3, UQCRB, ERBB4, IP3, or MCU. In certain embodiments, the cancer expresses elevated levels of SLC16A7, ELF3, or NROB1.

In certain embodiments, the cancer is characterized by elevated functional activity by the target. In certain embodiments, the cancer is characterized by functional activity by the target that is at least 10%, 20%, 30%, 40%, 50%, 60%, 70%, 80%, 90%, or 100% greater than average.

In certain embodiments, the cancer is characterized by elevated functional activity of NOX5. In certain embodiments, the cancer is characterized by elevated functional activity of SLC7A11. In certain embodiments, the cancer is characterized by elevated functional activity of GSH. In certain embodiments, the cancer is characterized by elevated functional activity of GPX. In certain embodiments, the cancer is characterized by elevated functional activity of CYBA. In certain embodiments, the cancer is characterized by elevated functional activity of EDG2. In certain embodiments, the cancer is characterized by elevated functional activity of PPP1CC. In certain embodiments, the cancer is characterized by elevated functional activity of PP1C. In certain embodiments, the cancer is characterized by elevated functional activity of PP1gamma. In certain embodiments, the cancer is characterized by elevated functional activity of ROCK2. In certain embodiments, the cancer is characterized by elevated functional activity of RPS6KA2. In certain embodiments, the cancer is characterized by elevated functional activity of SYK. In certain embodiments, the cancer is characterized by elevated functional activity of AKT1. In certain embodiments, the cancer is characterized by elevated functional activity of AKT2. In certain embodiments, the cancer is characterized by elevated functional activity of BP. In certain embodiments, the cancer is characterized by elevated functional activity of GSR In certain embodiments, the cancer is characterized by elevated functional activity of G6PD. In certain embodiments, the cancer is characterized by elevated functional activity of ABCB5. In certain embodiments, the cancer is characterized by elevated functional activity of EPHA(i). In certain embodiments, the cancer is characterized by elevated functional activity of DLK1. In certain embodiments, the cancer is characterized by elevated functional activity of IDH1. In certain embodiments, the cancer is characterized by elevated functional activity of ME2/3. In certain embodiments, the cancer is characterized by elevated functional activity of HTATIP2. certain embodiments, the cancer is characterized by elevated functional activity of DKK1. In certain embodiments, the cancer is characterized by elevated functional activity of RAC3. In certain embodiments, the cancer is characterized by elevated functional activity of UQCRB. In certain embodiments, the cancer is characterized by elevated functional activity of ERBB4. In certain embodiments, the cancer is characterized by elevated functional activity of IP3. In certain embodiments, the cancer is characterized by elevated functional activity of MCU. In certain embodiments, the cancer is characterized by elevated functional activity of SLC16A7. In certain embodiments, the cancer is characterized by elevated functional activity of ELF3. In certain embodiments, the cancer is characterized by elevated functional activity of NROB1. In certain embodiments, the cancer is characterized by elevated functional activity of EPHA2.

In certain embodiments, the cancer is characterized by elevated functional activity of NOX5, SLC7A11, GSH, GPX, CYBA, EDG2, PPP1CC, PP1C, PP1gamma, ROCK2, RPS6KA2, SYK, BP, GSR, G6PD, ABCB5, EPHA(i), DLK1, IDH1, ME2/3, HTATIP2, DKK1 RAC3, UQCRB, ERBB4, IP3, MCU, SLC16A7, ELF3, NROB1 or EPHA2.

In certain embodiments, the cancer is characterized by elevated functional activity of NOX5, SLC7A11, GSH, or GPX. In certain embodiments, the cancer is characterized by elevated functional activity of CYBA, EDG2, PPP1CC, PP1C, or PP1gamma. In certain embodiments, the cancer is characterized by elevated functional activity of ROCK2, EPHA(i), or EPHA2. In certain embodiments, the cancer is characterized by elevated functional activity of RPS6KA2 or SYK. In certain embodiments, the cancer is characterized by elevated functional activity of AKT1 or AKT2. In certain embodiments, the cancer is characterized by elevated functional activity of BP, GSR, G6PD, or ABCB5. In certain embodiments, the cancer is characterized by elevated functional activity of DLK1, IDH1, ME2/3, or HTATIP2. In certain embodiments, the cancer is characterized by elevated functional activity of DKK1, RAC3, UQCRB, ERBB4, IP3, or MCU. In certain embodiments, the cancer is characterized by elevated functional activity of SLC16A7, ELF3, or NROB1.

In certain embodiments, the cancer is characterized by an elevated oxidation state. In certain embodiments, the cancer has an oxidation state that is at least 10% more oxidative than average. In certain embodiments, the cancer has an oxidation state that is at least 20% more oxidative than average. In certain embodiments, the cancer has an oxidation state that is at least 30% more oxidative than average. In certain embodiments, the cancer has an oxidation state that is at least 50% more oxidative than average. In certain embodiments, the cancer has an oxidation state that is at least 75% more oxidative than average. In certain embodiments, the cancer has an oxidation state that is at least 100% more oxidative than average.

### First Therapeutic Agent

The therapeutic method may be characterized according to the identity of the first therapeutic agent. For example, in certain embodiments, the first therapeutic agent comprises a small organic compound and/or an antibody. In certain embodiments, the first therapeutic agent comprises a small organic compound. In certain embodiments, the first therapeutic agent comprises an antibody. In certain embodiments, the first therapeutic comprises an antibody, antibody-drug conjugate, an oligonucleotide, or immunoglobulin scaffold (e.g., IgG scaffold).

In certain embodiments, the first therapeutic agent comprises a BRAF inhibitor. In certain embodiments, the first therapeutic agent comprises a MEK inhibitor. In certain embodiments, the first therapeutic agent comprises a BRAF inhibitor and a MEK inhibitor.

The therapeutic method may be characterized according to the BRAF inhibitor and/or MEK inhibitor administered to the patient. For example, in certain embodiments, the first therapeutic agent comprises a BRAF inhibitor. In certain embodiments, the first therapeutic agent comprises a BRAF inhibitor selected from the group consisting of dabrafenib, Plx4720, Raf265, vemurafenib, and a pharmaceutically acceptable salt of any of the foregoing. In certain embodiments, the first therapeutic agent is a BRAF inhibitor. In certain embodiments, the first therapeutic agent is a BRAF inhibitor selected from the group consisting of dabrafenib, Plx4720, Raf265, vemurafenib, and a pharmaceutically acceptable salt of any of the foregoing.

In certain embodiments, the first therapeutic agent comprises a MEK inhibitor. In certain embodiments, the first therapeutic agent comprises a MEK inhibitor selected from the group consisting of binimetinib, cobimetinib, Pd98059, selumetinib, trametinib, and a pharmaceutically acceptable salt of any of the foregoing. In certain embodiments, the first therapeutic agent is a MEK inhibitor. In certain embodiments, the first therapeutic agent is a MEK inhibitor selected from the group consisting of binimetinib, cobimetinib, Pd98059, selumetinib, trametinib, and a pharmaceutically acceptable salt of any of the foregoing.

In certain embodiments, the first therapeutic agent comprises (i) dabrafenib or a pharmaceutically acceptable salt thereof and (ii) trametinib or a pharmaceutically acceptable salt thereof. In certain embodiments, the first therapeutic agent comprises (i) vemurafenib or a pharmaceutically acceptable salt thereof and (ii) cobimetinib or a pharmaceutically acceptable salt thereof. In certain embodiments, the first therapeutic agent comprises (i) encorafenib or a pharmaceutically acceptable salt thereof and (ii) binimetinib or a pharmaceutically acceptable salt thereof.

### Second Therapeutic Agent

The therapeutic method may be characterized according to the identity of the second therapeutic agent. For example, in certain embodiments, the second therapeutic agent comprises a small organic compound and/or an antibody. In certain embodiments, the second therapeutic agent comprises a small organic compound. In certain embodiments, the second therapeutic agent comprises an antibody. In certain embodiments, the second therapeutic comprises an antibody, antibody-drug conjugate, an oligonucleotide, or immunoglobulin scaffold (e.g., IgG scaffold).

j The therapeutic method may be characterized according to the target inhibited by the second therapeutic agent. For example, in certain embodiments, the second therapeutic agent inhibits NOX5, SLC7A11, GSH, GPX, CYBA, EDG2, PPP1CC, PP1C, PP1gamma, ROCK2, RPS6KA2, SYK, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2.

In certain embodiments, the second therapeutic agent inhibits NOX5, SLC7A11, GSH, or GPX. In certain embodiments, the second therapeutic agent inhibits CYBA, EDG2, PPP1CC, PP1C, or PP1gamma. In certain embodiments, the second therapeutic agent inhibits ROCK2, EPHA(i), or EPHA2. In certain embodiments, the second therapeutic agent inhibits RPS6KA2 or SYK. In certain embodiments, the second therapeutic agent inhibits AKT1 or AKT2. In certain embodiments, the second therapeutic agent inhibits BP, GSR, G6PD, or ABCB5. In certain embodiments, the second therapeutic agent inhibits DLK1, IDH1, ME2/3, or HTATIP2. In certain embodiments, the second therapeutic agent inhibits DKK1, RAC3, UQCRB, ERBB4, IP3, or MCU. In certain embodiments, the second therapeutic agent inhibits SLC16A7, ELF3, or NROB1.

In certain embodiments, the second therapeutic agent inhibits NOX5. In certain embodiments, the second therapeutic agent is a NOX5 inhibitor selected from the group consisting of apocynin, VAS2870, Pep1, Pep3, melittin, diphenyleneiodonium, GKT136901, setanaxib (GKT137831, GKT83), and pharmaceutically acceptable salts of any of the foregoing. In certain embodiments, the second therapeutic agent is apocynin or a pharmaceutically acceptable salt thereof. In certain embodiments, the second therapeutic agent is VAS2870 or a pharmaceutically acceptable salt thereof. In certain embodiments, the second therapeutic agent is Pep1 or a pharmaceutically acceptable salt thereof. In certain embodiments, the second therapeutic agent is Pep3 or a pharmaceutically acceptable salt thereof. In certain embodiments, the second therapeutic agent is melittin or a pharmaceutically acceptable salt thereof. In certain embodiments, the second therapeutic agent is GKT136901 or a pharmaceutically acceptable salt thereof. In certain embodiments, the second therapeutic agent is setanaxib or a pharmaceutically acceptable salt thereof. In certain embodiments, the second therapeutic agent is diphenyleneiodonium or a pharmaceutically acceptable salt thereof. In certain embodiments, the second therapeutic agent is diphenyieneiodonium chloride. In certain embodiments, the second therapeutic agent is a NOX5 inhibitor disclosed in one of the following patent applications, each of which is incorporated by reference in their entirety: WO2008113856A1, WO2010035220A1, WO2010035217A1, WO2010035219A2, WO2010035221A1, WO2016098005A1, and WO2019086579 A1.

In certain embodiments, the second therapeutic agent inhibits SLC7A11. In certain embodiments, the second therapeutic agent is a SLC7A11 inhibitor selected from the group consisting of sulfasalazine or a pharmaceutically acceptable salt thereof, erastin or a pharmaceutically acceptable salt thereof, an AgilVax vaccine, and an AgilVax antibody. In certain embodiments, the second therapeutic agent is sulfasalazine or a pharmaceutically acceptable salt thereof. In certain embodiments, the second therapeutic agent is erastin or a pharmaceutically acceptable salt thereof. In certain embodiments, the second therapeutic agent is an AgilVax vaccine. In certain embodiments, the second therapeutic agent is an AgilVax antibody. In certain embodiments, the second therapeutic agent is an antibody-drug conjugate comprising an SLC7A11 inhibitor. In certain embodiments, the second therapeutic agent is an antibody-drug conjugate comprising an AgilVax antibody.

In certain embodiments, the second therapeutic agent inhibits GSH. In certain embodiments, the second therapeutic agent is a GSH inhibitor selected from the group consisting of buthionine sulfoximine and a pharmaceutically acceptable salt thereof.

In certain embodiments, the second therapeutic agent inhibits GPX. In certain embodiments, the second therapeutic agent is a GPX inhibitor selected from the group consisting of tiopronin, mercaptosuccinic acid (MSA), misonidazole, penicillamine, buthionine sulfoximine, gold(I) thioglucose, and pharmaceutically acceptable salts of any of the foregoing. In certain embodiments, the second therapeutic agent is tiopronin, or a pharmaceutically acceptable salt thereof. In certain embodiments, the second therapeutic agent is mercaptosuccinic acid, or a pharmaceutically acceptable salt thereof. In certain embodiments, the second therapeutic agent is misonidazole, or a pharmaceutically acceptable salt thereof. In certain embodiments, the second therapeutic agent is penicillamine, or a pharmaceutically acceptable salt thereof. In certain embodiments, the second therapeutic agent is buthionine sulfoximine, or a pharmaceutically acceptable salt thereof. In certain embodiments, the second therapeutic agent is gold(I) thioglucose, or a pharmaceutically acceptable salt thereof.

In certain embodiments, the second therapeutic agent inhibits CYBA. In certain embodiments, the second therapeutic agent is a CYBA inhibitor selected from dextromethorphan and pharmaceutically acceptable salts thereof. In certain embodiments, the second therapeutic agent is a CYBA inhibitor disclosed in WO2009044392A2, which is incorporated by reference in its entirety.

In certain embodiments, the second therapeutic agent inhibits EDG2. In certain embodiments, the second therapeutic agent is an EDG2 inhibitor selected from the group consisting of SU6656, LY294002, wortmannin, Nm23-H1, tetradecyl phosphonate, Kil6425, and pharmaceutically acceptable salts of any of the foregoing. In certain embodiments, the second therapeutic agent is SU6656, or a pharmaceutically acceptable salt thereof. In certain embodiments, the second therapeutic agent is LY294002, or a pharmaceutically acceptable salt thereof. In certain embodiments, the second therapeutic agent is wortmannin, or a pharmaceutically acceptable salt thereof. In certain embodiments, the second therapeutic agent is Nm23-H1, or a pharmaceutically acceptable salt thereof. In certain embodiments, the second therapeutic agent is tetradecyl phosphonate, or a pharmaceutically acceptable salt thereof. In certain embodiments, the second therapeutic agent is Kil6425, or a pharmaceutically acceptable salt thereof. In certain embodiments, the second therapeutic agent is an EDG2 inhibitor disclosed in WO2012007632A9, which is incorporated by reference in its entirety.

In certain embodiments, the second therapeutic agent inhibits PPP1CC. In certain embodiments, the second therapeutic agent is a PPP1CC inhibitor selected from the group consisting of ceramide, okadaic acid, calyculin A, canthardic acid, DL-2-Amino-3-phosphonopropionic acid (AP3), tautomycin, tautomycetin, fostriecin, microcystin-LR, cantharidin, thyrsiferyl-23-acetate, nodularin, dephostatin, 3,4-dephostatin, fumonisin B1, motuporin, and pharmaceutically acceptable salts of any of the foregoing. In certain embodiments, the second therapeutic agent is ceramide, or a pharmaceutically acceptable salt thereof. In certain embodiments, the second therapeutic agent is okadaic acid, or a pharmaceutically acceptable salt thereof. In certain embodiments, the second therapeutic agent is calyculin A, or a pharmaceutically acceptable salt thereof. In certain embodiments, the second therapeutic agent is canthardic acid, or a pharmaceutically acceptable salt thereof. In certain embodiments, the second therapeutic agent is DL-2-Amino-3-phosphonopropionic acid, or a pharmaceutically acceptable salt thereof. In certain embodiments, the second therapeutic agent is tautomycin, or a pharmaceutically acceptable salt thereof. In certain embodiments, the second therapeutic agent is tautonlycetin, or a pharmaceutically acceptable salt thereof. In certain embodiments, the second therapeutic agent is fostriecin, or a pharmaceutically acceptable salt thereof. In certain embodiments, the second therapeutic agent is microcystin-LR, or a pharmaceutically acceptable salt thereof. In certain embodiments, the second therapeutic agent is cantharidin, or a pharmaceutically acceptable salt thereof. In certain embodiments, the second therapeutic agent is thyrsiferyl-23-acetate, or a pharmaceutically acceptable salt thereof. In certain embodiments, the second therapeutic agent is nodularin, or a pharmaceutically acceptable salt thereof. In certain embodiments, the second therapeutic agent is dephostatin, or a pharmaceutically acceptable salt thereof. In certain embodiments, the second therapeutic agent is 3,4-dephostatin, or a pharmaceutically acceptable salt thereof. In certain embodiments, the second therapeutic agent is fumonisin B1, or a pharmaceutically acceptable salt thereof. In certain embodiments, the second therapeutic agent is motuporin, or a pharmaceutically acceptable salt thereof.

In certain embodiments, the second therapeutic agent inhibits PP1C. In certain embodiments, the second therapeutic agent is a PP1C inhibitor selected from the group consisting of ceramide, okadaic acid, calyculin A, canthardic acid, DL-2-Amino-3-phosphonopropionic acid (AP3), tautomycin, tautomycetin, fostriecin, microcystin-LR, cantharidin, thyrsiferyl-23-acetate, nodularin, dephostatin, 3,4-dephostatin, fumonisin B1, motuporin, and pharmaceutically acceptable salts of any of the foregoing. In certain embodiments, the second therapeutic agent is ceramide, or a pharmaceutically acceptable salt thereof. In certain embodiments, the second therapeutic agent is okadaic acid, or a pharmaceutically acceptable salt thereof. In certain embodiments, the second therapeutic agent is calyculin A, or a pharmaceutically acceptable salt thereof. In certain embodiments, the second therapeutic agent is canthardic acid, or a pharmaceutically acceptable salt thereof. In certain embodiments, the second therapeutic agent is DL-2-Amino-3-phosphonopropionic acid, or a pharmaceutically acceptable salt thereof. In certain embodiments, the second therapeutic agent is tautomycin, or a pharmaceutically acceptable salt thereof. In certain embodiments, the second therapeutic agent is tautomycetin, or a pharmaceutically acceptable salt thereof. In certain embodiments, the second therapeutic agent is fostriecin, or a pharmaceutically acceptable salt thereof. In certain embodiments, the second therapeutic agent is microcystin-LR, or a pharmaceutically acceptable salt thereof. In certain embodiments, the second therapeutic agent is cantharidin, or a pharmaceutically acceptable salt thereof. In certain embodiments, the second therapeutic agent is thyrsiferyl-23-acetate, or a pharmaceutically acceptable salt thereof. In certain embodiments, the second therapeutic agent is nodularin, or a pharmaceutically acceptable salt thereof. In certain embodiments, the second therapeutic agent is dephostatin, or a pharmaceutically acceptable salt thereof. In certain embodiments, the second therapeutic agent is 3,4-dephostatin, or a pharmaceutically acceptable salt thereof. In certain embodiments, the second therapeutic agent is fumonisin B1, or a pharmaceutically acceptable salt thereof. In certain embodiments, the second therapeutic agent is motuporin, or a pharmaceutically acceptable salt thereof.

In certain embodiments, the second therapeutic agent inhibits PP1gamma. In certain embodiments, the second therapeutic agent is a PP1 gamma inhibitor selected from the group consisting of staurosporin, okadaic acid, calyculin A, canthardic acid, DL-2-Amino-3-phosphonopropionic acid (AP3), tautomycin, tautomycetin, fostriecin, microcystin-LR, cantharidin, thyrsiferyl-23-acetate, nodularin, dephostatin, 3,4-dephostatin, fumonisin B1, motuporin, and pharmaceutically acceptable salts of any of the foregoing. In certain embodiments, the second therapeutic agent is staurosporin, or a pharmaceutically acceptable salt thereof. In certain embodiments, the second therapeutic agent is okadaic acid, or a pharmaceutically acceptable salt thereof. In certain embodiments, the second therapeutic agent is calyculin A, or a pharmaceutically acceptable salt thereof. In certain embodiments, the second therapeutic agent is canthardic acid, or a pharmaceutically acceptable salt thereof. In certain embodiments, the second therapeutic agent is DL-2-Amino-3-phosphonopropionic acid, or a pharmaceutically acceptable salt thereof. In certain embodiments, the second therapeutic agent is tautomycin, or a pharmaceutically acceptable salt thereof. In certain embodiments, the second therapeutic agent is tautomycetin, or a pharmaceutically acceptable salt thereof. In certain embodiments, the second therapeutic agent is fostriecin, or a pharmaceutically acceptable salt thereof. In certain embodiments, the second therapeutic agent is microcystin-LR, or a pharmaceutically acceptable salt thereof. In certain embodiments, the second therapeutic agent is cantharidin, or a pharmaceutically acceptable salt thereof. In certain embodiments, the second therapeutic agent is thyrsiferyl-23-acetate, or a pharmaceutically acceptable salt thereof. In certain embodiments, the second therapeutic agent is nodularin, or a pharmaceutically acceptable salt thereof. In certain embodiments, the second therapeutic agent is dephostatin, or a pharmaceutically acceptable salt thereof. In certain embodiments, the second therapeutic agent is 3,4-dephostatin, or a pharmaceutically acceptable salt thereof. In certain embodiments, the second therapeutic agent is fumonisin B1, or a pharmaceutically acceptable salt thereof. In certain embodiments, the second therapeutic agent is motuporin, or a pharmaceutically acceptable salt thereof.

In certain embodiments, the second therapeutic agent inhibits ROCK2. In certain embodiments, the second therapeutic agent is a ROCK2 inhibitor selected from the group consisting of Fasudil (HA-1077), KD025, GSK429286A, thiazovivin, AS-1892802, Y-27632, 1-((5-chloronaphthalen-1-yl)sulfonyl)-1,4-diazepane, N-(2-(2-(dimethylamino)ethoxy)-4-(1H-pyrazol-4-yl)phenyl)-2,3dihydroberizo[b][1,4]dioxine-2-carboxamide, and pharmaceutically acceptable salts of any of the foregoing. In certain embodiments, the second therapeutic agent is Fasudil, or a pharmaceutically acceptable salt thereof. In certain embodiments, the second therapeutic agent is KD025, or a pharmaceutically acceptable salt thereof. In certain embodiments, the second therapeutic agent is GSK429286A, or a pharmaceutically acceptable salt thereof. In certain embodiments, the second therapeutic agent is thiazovivin, or a pharmaceutically acceptable salt thereof. In certain embodiments, the second therapeutic agent is AS-1892802, or a pharmaceutically acceptable salt thereof. In certain embodiments, the second therapeutic agent is Y-27632, or a pharmaceutically acceptable salt thereof. In certain embodiments, the second therapeutic agent is 1-((5-chloronaphthalen-1-yl)sulfonyl)-1,4-diazepane, or a pharmaceutically acceptable salt thereof. In certain embodiments, the second therapeutic agent is N-(2-(2-(dimethylamino)ethoxy)-4-(1H-pyrazol-4-yl)phenyl)-2,3dihydrobenzo[b][1,4]dioxine-2-carboxamide, or a pharmaceutically acceptable salt thereof. In certain embodiments, the second therapeutic agent is a ROCK2 inhibitor disclosed in one of the following patent applications, each of which is incorporated by reference in their entirety: WO2015054317A1, WO2009158587A1, WO2010104851A1, WO2008054599A2, WO2014055996A2, WO2014134391A1, and WO2019194598A1.

In certain embodiments, the second therapeutic agent inhibits RPS6KA2. In certain embodiments, the second therapeutic agent is a RPS6KA2 inhibitor selected from the group consisting of BRD 7389, BI-D1870, AT9283, and pharmaceutically acceptable salts of any of the foregoing. In certain embodiments, the second therapeutic agent is BRD 7389, or a pharmaceutically acceptable salt thereof. In certain embodiments, the second therapeutic agent is BI-D1870, or a pharmaceutically acceptable salt thereof. In certain embodiments, the second therapeutic agent is AT9283, or a pharmaceutically acceptable salt thereof.

In certain embodiments, the second therapeutic agent inhibits SYK. In certain embodiments, the second therapeutic agent is an SYK inhibitor selected from the group consisting of GSK 143, piceatannol, R406, fostamatinib, entospletinib (GS-9973), cerdulatinib (PRT062070), TAK-659, and pharmaceutically acceptable salts of any of the foregoing. In certain embodiments, the second therapeutic agent is GSK 143 or a pharmaceutically acceptable salt thereof. In certain embodiments, the second therapeutic agent is piceatannol or a pharmaceutically acceptable salt thereof. In certain embodiments, the second therapeutic agent is R406 or a pharmaceutically acceptable salt thereof. In certain embodiments, the second therapeutic agent is fostamatinib or a pharmaceutically acceptable salt thereof. In certain embodiments, the second therapeutic agent is entospletinib or a pharmaceutically acceptable salt thereof. In certain embodiments, the second therapeutic agent is cerdulatinib or a pharmaceutically acceptable salt thereof. In certain embodiments, the second therapeutic agent is TAK-659 or a pharmaceutically acceptable salt thereof.

In certain embodiments, the second therapeutic agent inhibits AKT1. In certain embodiments, the second therapeutic agent is an AKT1 inhibitor selected from the group consisting of A-674563, AZD5363, perifosine, afuresertib, uprosertib, triciribine, MK-2206, GSK690693, AT7867, ipatasertib (GDC-0068), AT13148, and pharmaceutically acceptable salts of any of the foregoing. In certain embodiments, the second therapeutic agent is A-674563, or a pharmaceutically acceptable salt thereof. In certain embodiments, the second therapeutic agent is AZD5363, or a pharmaceutically acceptable salt thereof. In certain embodiments, the second therapeutic agent is perifosine, or a pharmaceutically acceptable salt thereof. In certain embodiments, the second therapeutic agent is afuresertib, or a pharmaceutically acceptable salt thereof. In certain embodiments, the second therapeutic agent is uprosertib, or a pharmaceutically acceptable salt thereof. In certain embodiments, the second therapeutic agent is triciribine, or a pharmaceutically acceptable salt thereof. In certain embodiments, the second therapeutic agent is MK-2206, or a pharmaceutically acceptable salt thereof. In certain embodiments, the second therapeutic agent is GSK690693, or a pharmaceutically acceptable salt thereof. In certain embodiments, the second therapeutic agent is AT7867, or a pharmaceutically^{,} acceptable salt thereof. In certain embodiments, the second therapeutic agent is ipatasertib, or a pharmaceutically acceptable salt thereof. In certain embodiments, the second therapeutic agent is AT113148, or a pharmaceutically acceptable salt thereof. In certain embodiments, the second therapeutic agent is an AKT1 inhibitor disclosed in WO2008098104A1, which is incorporated by reference in its entirety. In certain embodiments, the second therapeutic agent is an AKT1 inhibitor disclosed in Nitulescu, et al., Int J Oncol. 2016 March; 48(3): 869-885, which is incorporated by reference in its entirety.

In certain embodiments, the second therapeutic agent inhibits AKT2. In certain embodiments, the second therapeutic agent is an AKT2 inhibitor selected from the group consisting of CCT128930, AZD5363, perifosine, afuresertib, uprosertib, triciribine, MK-2206, GSK690693, AT7867, ipatasertib (GDC-0068), AT13148, and pharmaceutically acceptable salts of any of the foregoing. In certain embodiments, the second therapeutic agent is CCT128930, or a pharmaceutically acceptable salt thereof. In certain embodiments, the second therapeutic agent is AZD5363, or a pharmaceutically acceptable salt thereof. In certain embodiments, the second therapeutic agent is perifosine, or a pharmaceutically acceptable salt thereof. In certain embodiments, the second therapeutic agent is afuresertib, or a pharmaceutically acceptable salt thereof. In certain embodiments, the second therapeutic agent is uprosertib, or a pharmaceutically acceptable salt thereof. In certain embodiments, the second therapeutic agent is triciribine, or a pharmaceutically acceptable salt thereof. In certain embodiments, the second therapeutic agent is MK-2206, or a pharmaceutically acceptable salt thereof. In certain embodiments, the second therapeutic agent is GSK690693, or a pharmaceutically acceptable salt thereof. In certain embodiments, the second therapeutic agent is AT7867, or a pharmaceutically acceptable salt thereof. In certain embodiments, the second therapeutic agent is ipatasertib, or a pharmaceutically acceptable salt thereof. In certain embodiments, the second therapeutic agent is AT13148, or a pharmaceutically acceptable salt thereof. In certain embodiments, the second therapeutic agent is an AKT2 inhibitor disclosed in WO2008098104A1, which is incorporated by reference in its entirety. In certain embodiments, the second therapeutic agent is an AKT2 inhibitor disclosed in Nitulescu, et al., Int J Oncol. 2016 March; 48(3): 869-885, which is incorporated by reference in its entirety.

In certain embodiments, the second therapeutic agent inhibits BP. In certain embodiments, the second therapeutic agent is a BP inhibitor.

In certain embodiments, the second therapeutic agent inhibits GSR. In certain embodiments, the second therapeutic agent is a GSR inhibitor selected from the group consisting of carmustine, LY 83583, butein, 2-AAPA, acylfulvene and analogues thereof, NSC130362 and analogues thereof, and pharmaceutically acceptable salts of any of the foregoing. In certain embodiments, the second therapeutic agent is a GSR inhibitor selected from the group consisting of carmustine, LY 83583, butein, 2-AAPA, acylfulvene, NSC130362, and pharmaceutically acceptable salts of any of the foregoing. In certain embodiments, the second therapeutic agent is carmustine, or a pharmaceutically acceptable salt thereof. In certain embodiments, the second therapeutic agent is LY 83583, or a pharmaceutically acceptable salt thereof. In certain embodiments, the second therapeutic agent is butein, or a pharmaceutically acceptable salt thereof. In certain embodiments, the second therapeutic agent is 2-AAPA, or a pharmaceutically acceptable salt thereof. In certain embodiments, the second therapeutic agent is acylfulvene, or a pharmaceutically acceptable salt thereof. In certain embodiments, the second therapeutic agent is NSC130362, or a pharmaceutically acceptable salt thereof. In certain embodiments, the second therapeutic agent is a GSR inhibitor disclosed in Elliott, et al., Biochemical Pharmacology, Volume 44, Issue 8, 20 Oct. 1992, Pages 1603-1608.

In certain embodiments, the second therapeutic agent inhibits G6PD. In certain embodiments, the second therapeutic agent is a G6PD inhibitor selected from the group consisting of dantrolene, pyridoxal 5′-phosphate, 1 fluoro-2,4 dinitrobenzene, dehydroepiandrosterone (DHEA), epiandrosterone, and pharmaceutically acceptable salts of any of the foregoing. In certain embodiments, the second therapeutic agent is dantrolene, or a pharmaceutically acceptable salt thereof. In certain embodiments, the second therapeutic agent is pyridoxal 5′-phosphate, or a pharmaceutically acceptable salt thereof. In certain embodiments, the second therapeutic agent is 1 fluoro-2,4 dinitrobenzene, or a pharmaceutically acceptable salt thereof. In certain embodiments, the second therapeutic agent is dehydroepiandrosterone, or a pharmaceutically acceptable salt thereof. In certain embodiments, the second therapeutic agent is epiandrosterone, or a pharmaceutically acceptable salt thereof. In certain embodiments, the second therapeutic agent is a G6PD inhibitor disclosed in one of the following patent applications, each of which is incorporated by reference in their entirety: WO2009009523A2, WO2018093856A1, U.S. Pat. No. 9,381,193B2, and WO2006083051A1. In certain embodiments, the second therapeutic agent is a G6PD inhibitor disclosed in Hamilton, et al., J Med. Chem. 2012, 55, 9, 4431-4445, which is incorporated by reference in its entirety.

In certain embodiments, the second therapeutic agent inhibits ABCB5. in certain embodiments, the second therapeutic agent is an ABCB5 inhibitor disclosed in one of the following patent applications, each of which is incorporated by reference in their entirety: WO2010065711 A1, WO2016179576A1, and WO2008127656A1.

In certain embodiments, the second therapeutic agent inhibits EPHA(i). In certain embodiments, the second therapeutic agent is an EPHA(i) inhibitor selected from the group consisting of saracatinib (AZD0530), and pharmaceutically acceptable salts thereof. In certain embodiments, the second therapeutic agent is saracatinib, or a pharmaceutically acceptable salt thereof.

In certain embodiments, the second therapeutic agent inhibits DLK1. In certain embodiments, the second therapeutic agent is a DLK1 inhibitor.

In certain embodiments, the second therapeutic agent inhibits IDH1. In certain embodiments, the second therapeutic agent is an IDH1 inhibitor selected from the group consisting of ivosidenib, enasidenib, vorasidenib, BAY-1436032, AGI-5198, IDH305, FT-2102, HMS-101, MRK-A, GSK321, DS-1001b, and pharmaceutically acceptable salts of any of the foregoing. In certain embodiments, the second therapeutic agent is ivosidenib or a pharmaceutically acceptable salt thereof. In certain embodiments, the second therapeutic agent is enasidenib or a pharmaceutically acceptable salt thereof. In certain embodiments, the second. therapeutic agent is vorasidenib or a pharmaceutically acceptable salt thereof. In certain embodiments, the second therapeutic agent is BAY-1436032, or a pharmaceutically acceptable salt thereof. In certain embodiments, the second therapeutic agent is AGI-5198, or a pharmaceutically acceptable salt thereof. In certain embodiments, the second therapeutic agent is IDH305, or a pharmaceutically acceptable salt thereof. In certain embodiments, the second therapeutic agent is FT-2102, or a pharmaceutically acceptable salt thereof. In certain embodiments, the second therapeutic agent is HMS-101, or a pharmaceutically acceptable salt thereof. In certain embodiments, the second therapeutic agent is MRK-A, or a pharmaceutically^{,} acceptable salt thereof. In certain embodiments, the second therapeutic agent is GSK321, or a pharmaceutically acceptable salt thereof. In certain embodiments, the second therapeutic agent is DS-1001b, or a pharmaceutically acceptable salt thereof. In certain embodiments, the second therapeutic agent is an IDH1 inhibitor disclosed in one of the following patent applications, each of which is incorporated by reference in their entirety: WO2019222553A1, WO2017146795A1, WO2016106331 A1, WO2015121210A1, WO2012171506A1, and WO2011050210A1.

In certain embodiments, the second therapeutic agent inhibits ME2/3. In certain embodiments, the second therapeutic agent is a ME2/3 inhibitor selected from the group consisting of NPD389, embonic acid, and pharmaceutically acceptable salts of either of the foregoing. In certain embodiments, the second therapeutic agent is NPD389, or a pharmaceutically acceptable salt thereof. In certain embodiments, the second therapeutic agent is embonic acid, or a pharmaceutically acceptable salt thereof.

In certain embodiments, the second therapeutic agent inhibits HTATIP2. In certain embodiments, the second therapeutic agent is an HTATIP2 inhibitor selected from the group consisting of DB11077, and pharmaceutically acceptable salts thereof. In certain embodiments, the second therapeutic agent is DB11077, or a pharmaceutically acceptable salt thereof.

In certain embodiments, the second therapeutic agent inhibits DKK1. In certain embodiments, the second therapeutic agent is a DKK1 inhibitor selected from the group consisting of gallocyanine, NCI8642, and pharmaceutically acceptable salts of either of the foregoing. In certain embodiments, the second therapeutic agent is gallocyanine, or a pharmaceutically acceptable salt thereof. In certain embodiments, the second therapeutic agent is NCI8642, or a pharmaceutically acceptable salt thereof. In certain embodiments, the second therapeutic agent is a DKK1 inhibitor disclosed in Thysiadis, et al., Bioorganic Chem. 2018, 80, 230-244, which is incorporated by reference in its entirety.

In certain embodiments, the second therapeutic agent inhibits RAC3. In certain embodiments, the second therapeutic agent is an RAC3 inhibitor selected from the group consisting of EHT 1864, EHop-016, NSC23766, and pharmaceutically acceptable salts of any of the foregoing. In certain embodiments, the second therapeutic agent is EHT 1864, or a pharmaceutically acceptable salt thereof. In certain embodiments, the second therapeutic agent is EHop-016, or a pharmaceutically acceptable salt thereof. In certain embodiments, the second therapeutic agent is NSC23766, or a pharmaceutically acceptable salt thereof. In certain embodiments, the second therapeutic agent is a RAC3 inhibitor disclosed in one of the following patent applications, each of which is incorporated by reference in their entirety: WO2019023315A2 and US20130172552A1.

In certain embodiments, the second therapeutic agent inhibits UQCRB. In certain embodiments, the second therapeutic agent is an UQCRB inhibitor selected from the group consisting of 6-((1-Hydroxynaphthalen-4-ylamino)dioxysulfone)-2H-naphtho[1,8-bc]thiophen-2-one (HDNT), or a pharmaceutically acceptable salt thereof. In certain embodiments, the second therapeutic agent is a UQCRB inhibitor disclosed in Jung, et al., J. Med. Chem. 2014, 57, 19, 7990-7998, which is incorporated by reference in its entirety.

In certain embodiments, the second therapeutic agent inhibits ERBB4. In certain embodiments, the second therapeutic agent is an ERBB4 inhibitor selected from the group consisting of AEE788 (NVP-AEE788), AC480 (BMS-599626), ibrutinib, lapatinib, neratinib, afatinib, osimertinib, dacomitinib, avitinib, olmutinib, allitinib, TAK-285, TAS6417, poziotinib, AZ5104, pelitinib, and pharmaceutically acceptable salts of any of the foregoing. In certain embodiments, the second therapeutic agent is AEE788 (NVP-AEE788) or a pharmaceutically acceptable salt thereof. In certain embodiments, the second therapeutic agent is AC480 (BMS-599626) or a pharmaceutically acceptable salt thereof. In certain embodiments, the second therapeutic agent is ibrutinib or a pharmaceutically acceptable salt thereof. In certain embodiments, the second therapeutic agent is lapatinib or a pharmaceutically acceptable salt thereof. In certain embodiments, the second therapeutic agent is neratinib or a pharmaceutically acceptable salt thereof. In certain embodiments, the second therapeutic agent is afatinib or a pharmaceutically acceptable salt thereof. In certain embodiments, the second therapeutic agent is osimertinib or a pharmaceutically acceptable salt thereof. In certain embodiments, the second therapeutic agent is dacomitinib or a pharmaceutically acceptable salt thereof. In certain embodiments, the second therapeutic agent is avitinib or a pharmaceutically acceptable salt thereof. In certain embodiments, the second therapeutic agent is olmutinib or a pharmaceutically acceptable salt thereof. In certain embodiments, the second therapeutic agent is allitinib or a pharmaceutically acceptable salt thereof. In certain embodiments, the second therapeutic agent is TAK-285 or a pharmaceutically acceptable salt thereof. In certain embodiments, the second therapeutic agent is TAS6417 or a pharmaceutically acceptable salt thereof. In certain embodiments, the second therapeutic agent is poziotinib or a pharmaceutically acceptable salt thereof. In certain embodiments, the second therapeutic agent is AZ5104 or a pharmaceutically acceptable salt thereof. In certain embodiments, the second therapeutic agent is pelitinib or a pharmaceutically acceptable salt thereof. In certain embodiments, the second therapeutic agent is a MCU inhibitor disclosed in one of the following patent applications, each of which is incorporated by reference in their entirety: WO2008005983, WO2003012072, WO2003070912, US20060233808, and US20060128636.

In certain embodiments, the second therapeutic agent inhibits IP3. In certain embodiments, the second therapeutic agent is an IP3 inhibitor selected from the group consisting of Araguspongin B, cAMP, Xestospongin C, 2-aminoethoxydiphenyl borate, and pharmaceutically acceptable salts of any of the foregoing. In certain embodiments, the second therapeutic agent is Araguspongin B, or a pharmaceutically acceptable salt thereof. In certain embodiments, the second therapeutic agent is cAMP, or a pharmaceutically acceptable salt thereof. In certain embodiments, the second therapeutic agent is Xestospongin C or a pharmaceutically acceptable salt thereof. In certain embodiments, the second therapeutic agent is 2-aminoethoxydiphenyl borate or a pharmaceutically acceptable salt thereof.

In certain embodiments, the second therapeutic agent inhibits MCU. In certain embodiments, the second therapeutic agent is a MCU inhibitor selected from the group consisting of DS16570511, Ru360, Ru265, KB-R7943, and pharmaceutically acceptable salts of any of the foregoing. In certain embodiments, the second therapeutic agent is DS16570511, or a pharmaceutically acceptable salt thereof. In certain embodiments, the second therapeutic agent is Ru360, or a pharmaceutically acceptable salt thereof. In certain embodiments, the second therapeutic agent is Ru265, or a pharmaceutically acceptable salt thereof. In certain embodiments, the second therapeutic agent is KB-R7943, or a pharmaceutically acceptable salt thereof. In certain embodiments, the second therapeutic agent is KB-R7943 mesylate. In certain embodiments, the second therapeutic agent is a MCU inhibitor disclosed in US20190247427A1, which is incorporated by reference in its entirety.

In certain embodiments, the second therapeutic agent inhibits EPHA2. In certain embodiments, the second therapeutic agent is an EPHA2 inhibitor selected from the group consisting of OSI-027, saracatinib (AZD0530), dasatinib, bosutinib, and pharmaceutically acceptable salts of any of the foregoing. In certain embodiments, the second therapeutic agent is OSI-027, or a pharmaceutically acceptable salt thereof. In certain embodiments, the second. therapeutic agent is saracatinib, or a pharmaceutically acceptable salt thereof. In certain embodiments, the second therapeutic agent is dasatinib, or a pharmaceutically acceptable salt thereof. In certain embodiments, the second therapeutic agent is bosutinib, or a pharmaceutically acceptable salt thereof.

In certain embodiments, the second therapeutic agent inhibits SLC16A7. In certain embodiments, the second therapeutic agent is an SLC16A7 inhibitor selected from the group consisting of 7ACC2 and pharmaceutically acceptable salts thereof.

In certain embodiments, the second therapeutic agent inhibits ELF3. In certain embodiments, the second therapeutic agent is an ELF3 inhibitor.

In certain embodiments, the second therapeutic agent inhibits NROB1. In certain embodiments, the second therapeutic agent is an NROB1 inhibitor.

### Patients for Treatment

The therapeutic method may be further characterized according to the patient to be treated. In certain embodiments, the patient is an adult human. In certain embodiments, the cancer has been in remission for at least one month, at the time of treating the patient. In certain embodiments, the patient has not previously received a BRAF inhibitor for treatment of the cancer. In certain embodiments, the cancer is characterized by elevated expression levels of the target. In certain embodiments, the cancer is characterized by elevated functional activity by the target. In certain embodiments, the cancer is characterized by an elevated oxidation state.

In certain embodiments, the method further comprises selecting the patient for treatment by analyzing a sample from the patient to identify at least one of:
a. elevated expression levels of at least one target;
b. elevated functional activity of at least one target; or
c. elevated oxidation state of the cancer.

### Therapeutic Effect

The therapeutic method may be further characterized according to the therapeutic effect and outcome of the treatment method on the patient. For example, in certain embodiments, there is at least a 20% reduction in the size of at least one localized cancer in the patient. In certain embodiments, there is at least a 35% reduction in the size of at least one localized cancer in the patient. In certain embodiments, there is at least a 50% reduction in the size of at least one localized cancer in the patient.

In certain embodiments, there is at least a 20% reduction in the distribution of the cancer in the patient. In certain embodiments, there is at least a 35% reduction in the distribution of the cancer in the patient. In certain embodiments, there is at least a 50% reduction in the distribution of the cancer in the patient.

### Dose, Timing, and Route tog Administration of the First and Second Therapeutic Agents

The therapeutic method may be characterized according to features of administration of the first therapeutic agent. For instance, in certain embodiments, the therapeutic method may be characterized according to the dose of the first therapeutic agent administered to the patient. Accordingly, in certain embodiments, the first therapeutic agent is administered at a dosage ranging from about 0.01 mg to about 0.1 mg, about 0.1 mg to about 0.5 mg, about 0.5 mg to about 1 mg, about 1 mg to about 3 mg, about 3 mg to about 5 mg, about 5 mg to about 10 mg, about 10 mg to about 20 mg, about 20 mg to about 30 mg, about 30 mg to about 50 mg, about 50 mg to about 75 mg, about 75 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, about 1000 mg to about 1500 mg, about 1500 mg to about 2000 mg, or greater than 2000 mg.

The therapeutic method may also be characterized according to the frequency of administration of the first therapeutic agent. For example, in certain embodiments, the first therapeutic agent is administered 1, 2, or 3 times per day. in certain embodiments, the first therapeutic agent is administered once per day.

The therapeutic method may be further characterized according to the route of administration of the first therapeutic agent. For example, in certain embodiments, the first therapeutic agent is administered by intravenous administration. in certain embodiments, the first therapeutic agent is administered by oral administration. in certain embodiments, the first therapeutic agent is administered by parenteral administration.

The therapeutic method may be characterized according to features of administration of the second therapeutic agent. For instance, in certain embodiments, the therapeutic method. may be characterized according to the dose of the second therapeutic agent administered to the patient. Accordingly, in certain embodiments, the second therapeutic agent is administered at a dosage ranging from about 0.01 mg to about 0.1 mg, about 0.1 mg to about 0.5 mg, about 0.5 mg to about 1 mg, about 1 mg to about 3 mg, about 3 mg to about 5 mg, about 5 mg to about 10 mg, about 10 mg to about 20 mg, about 20 mg to about 30 mg, about 30 mg to about 50 mg, about 50 mg to about 75 mg, about 75 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, about 1000 mg to about 1500 mg, about 1500 mg to about 2000 mg, or greater than 2000 mg.

The therapeutic method may also be characterized according to the frequency of administration of the second therapeutic agent. For example, in certain embodiments, the second therapeutic agent is administered 1, 2, or 3 times per day. In certain embodiments, the second therapeutic agent is administered once per day.

The therapeutic method may be further characterized according to the route of administration of the second therapeutic agent. For example, in certain embodiments, the second therapeutic agent is administered by intravenous administration. In certain embodiments, the second therapeutic agent is administered by oral administration. In certain embodiments, the second therapeutic agent is administered by parenteral administration.

### Exemplary Dosing Amounts & Treatment Cycles for the First and Second Therapeutic Agents

Generally, a therapeutic agent is delivered to the patient in a therapeutically effective amount. The therapeutically effective amount of a therapeutic agent may vary with the activity of the specific agent employed; the metabolic stability and length of action of that agent; the species, age, body weight, general health, dietary status, sex and diet of the subject; the mode and time of administration; rate of excretion; drug combination, if any; and extent of presentation and/or severity of the particular condition being treated. The precise dosage can be determined, may involve one or several administrations per day, in whichever order is necessary or desirable, to yield the desired results, and the dosage may be adjusted by the individual practitioner to achieve a desired effect. Preferably, the dosage amount of the agent(s) used should be sufficient to interact solely with tumor cells, leaving normal cells essentially unharmed.

The dosage amount may be administered in a single dose or in the form of individual divided doses, such as from one to four or more times per day. In the event that the response in a subject is insufficient at a certain dose, even higher doses (or effective higher doses by a different, more localized delivery route) may be employed to the extent of patient tolerance.

Components in a combination therapy may be administered in a particular order and/or according to a treatment cycle, For example, in certain embodiments, at least one dose of the first therapeutic agent is administered to the patient prior to administering the second therapeutic agent. In certain embodiments, at least one dose of the first therapeutic agent is administered to the patient prior to administering the second therapeutic agent, but while there remains an effective amount of the first therapeutic agent in the patient. In certain embodiments, all doses of the first therapeutic agent are administered to the patient prior to administering the second therapeutic agent. In certain embodiments, at least one dose of the second therapeutic agent is administered to the patient prior to administering the first therapeutic agent. In certain embodiments, at least one dose of the second therapeutic agent is administered to the patient prior to administering the first therapeutic agent, but while there remains an effective amount of the second therapeutic agent in the patient. In certain embodiments, all doses of the second therapeutic agent are administered to the patient prior to administering the first therapeutic agent. In certain other embodiments, active components of the combination therapy may be co-administered simultaneously. In certain other embodiments, active components of the combination therapy may be co-administered in a predetermined manner, ratio, and order of addition so as to comprise a treatment cycle. In certain other embodiments, treatment cycles may be repeated in order to maximize benefit to the patient,

### Methods for Selecting Patients for Treatment

Another aspect of the invention provides a method for selecting a patient for treatment of cancer, the method comprises analyzing a sample from the patient to identify at least one of:
a. elevated expression levels of at least one target;
b. elevated functional activity of at least one target; or
c. elevated oxidation state of the cancer;
   wherein the target is independently selected from the group consisting of NOX5, SLC7A11, GSH, GPX, CYBA, EDG2, PPP1CC, PP1C, PP1 gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 and EPHA2;
      wherein upon identification of one or more of (a) elevated expression levels of at least one target, (b) elevated functional activity of at least one target, or (c) elevated oxidation state of the cancer, the patient is selected for treatment using a first therapeutic agent and a second therapeutic agent to treat the cancer, wherein the first therapeutic agent comprises a BRA F inhibitor and/or a MEK inhibitor, and the second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2.

Elevated expression levels of at least one target, elevated functional activity of at least one target, and elevated oxidation state of the cancer may be evaluated by various methods known to one of skill in the art, such as, for example, methods described in the Examples. For example, elevated expression levels of at least one target may be evaluated, for example, using quantitative real-time PCR, according to the procedures described in Example 3. Elevated oxidation state of the cancer may be evaluated, for example, using a whole-cell, in vitro oxidation state assay with CellRox™ DeepRed reagent, according to the procedures described in Example B.

Embodiments described above in connection with methods for treatment are reiterated here.

### III. MEDICAL KITS

Another aspect of the invention provides medical kits containing a therapeutic agent and/or pharmaceutical composition described herein, along with instructions for using the kits to treat a cancer described herein. In certain embodiments, the medical kit comprises (i) a first therapeutic agent comprising a BRAF inhibitor and/or a MEK inhibitor; (ii) a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2; and (iii) instructions for treating a cancer using the first therapeutic agent in combination with the second therapeutic agent. The instructions may specify, for example, the route of administration for the first therapeutic agent and the second therapeutic agent, such as by oral, intravenous, or parenteral administration. The medical kit may be further characterized according to one or more of the features described herein.

The medical kit may be further characterized according to additional information contained in the instructions for treating a cancer using the first therapeutic agent in combination with the second therapeutic agent. For example, in certain embodiments, the instructions for treating a cancer using the first therapeutic agent in combination with the second therapeutic agent contain additional information characterizing the cancer. In certain embodiments, the instructions for treating a cancer using the first therapeutic agent in combination with the second therapeutic agent contain information regarding one or more of the genomic, transcriptomic, proteomic, and metabolomic profile of the cancer. In certain embodiments, the instructions contain information regarding the genomic profile of the cancer. In certain embodiments, the instructions contain information regarding the transcriptomic profile of the cancer. In certain embodiments, the instructions contain information regarding the proteomic profile of the cancer. In certain embodiments, the instructions contain information regarding the metabolomic profile of the cancer.

### IV. PHARMACEUTICAL COMPOSITIONS

Therapeutic agents described herein may be formulated as a pharmaceutical composition comprising one or more therapeutic agents and a pharmaceutically acceptable carrier. For example, the first therapeutic agent can be formulated as a pharmaceutical composition that, for example, optionally further contains a further anti-cancer agent. A pharmaceutical composition that contains both a first therapeutic agent and a second therapeutic agent may be referred to as a co-formulated composition.

In certain embodiments, the invention provides a pharmaceutical composition comprising:
a. BRAF inhibitor and/or a MEK inhibitor;
b. a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2; and
c. a pharmaceutically acceptable carrier.

Embodiments described above in connection with methods for treatment are reiterated here.

In certain embodiments of the present invention, a therapeutic agent may be formulated as a pharmaceutically-acceptable oil; liposome; oil-water or lipid-oil-water emulsion or nanoemulsion; liquid; or salt, crystalline form, or other solid form delivered in a tablet or capsule. To facilitate such formulations, the therapeutic agent may be combined with a pharmaceutically-acceptable carrier or excipient therefor. Examples of pharmaceutically-acceptable carriers are well known in the art and include those conventionally used in pharmaceutical compositions, such as salts, lipids, buffers, chelating agents, flavorants, colorants, preservatives, absorption promoters to enhance bioavailability, antimicrobial agents, and combinations thereof, optionally in combination with other therapeutic ingredients.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain other embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

The description above describes multiple aspects and embodiments of the invention, including therapeutic methods, pharmaceutical compositions, and medical kits. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and are not intended to limit the invention.

### Example A. In Vitro Assays to Detect Synergy in Melanoma Treatment

### Cell Culture

Cells are grown and cultured in DMEM media containing 2 mM glutamine, 4.5 g/L glucose, 10% FBS and no sodium pyruvate (catalog 11965-092), except where specified otherwise. Cells are split and seeded at ratios that allowed for splitting 1-2× per week. For proliferative experiments, the cells are plated the night before, then reagents/drugs are prepared in fresh media and added to the cells immediately before the start of the experiment the following day.

### Proliferation Assays

The cells are labeled lentivirally with a fluorescent, nuclear tag (Histone 2B monomeric Red Fluorescent Protein, H2BmRFP from AddGene), flow sorted for H2BmRFP positivity (top 10-15% brightest), and fluorescently counted under drug treatments. Cells are seeded into 96 well plates (1-5,000 cells per well) and drug treatments applied the following day, including DMSO or PBS control (all concentrations contained equal percentage of DMSO or PBS solvent). Images are taken every 8-12 hours with sufficient image alignment (montaging) in order to capture about 25-100 cells per well/treatment (over the course of the experiment, cell counts typically exceed 1,000 in DMSO or low drug concentration wells). Direct measurements of cell counts are made using Cellavista software and Image J macros. The images are filtered through these computer programs to track and label each cell, quantifying the number of cells in each time-stamped frame. Proliferation is platted as log2 normalized growth, using the initial cell count from the first image frame for normalization.

Dose-response curves are generated using a 2-fold dilution of single drugs at various concentrations ranked highest to zero (zero containing only the solvent the drug was dissolved in, being either DMSO or H₂O); all concentrations contained equal volumes of the solvent used. For combinations with PLX4720 (BRAF inhibitor), 8 μM PLX4720 was used as diluent, and serially diluted with the second drug from highest concentration to lowest. The drug-induced proliferation (DIP) rates (see, Harris, L. A., et al. “An unbiased metric of antiproliferative drug effect in vitro,” Nature Methods', 2016, vol. 13, p. 497-500.) are calculated using the slope of the log2-normalized population curves after 24 hrs.

### Example B. Measuring the Redox State of a Cancer Cell

Briefly, melanoma cells are seeded in 96-well plates and treated with inhibitors for a duration specified. Hydrogen Peroxide (H₂O₂) is used as a positive control. Cells are incubated with 1 mM H₂O₂ mixed with growth media for an hour before incubation with CellRox reagent. CellRox™ DeepRed Reagent is used according to the manufacturer's instructions. The cells are then stained with 5 μM CellRox™ DeepRed reagent by diluting the probe in complete growth media and incubating for 30 minutes at 37° C. in tissue-culture incubators. Cells are then washed with PBS three times and imaged through a 20× objective with a Cellavista HighEnd Bioimager (SynenTec Bio-Services, Munster, Germany). Total ROS intensity is quantified by image segmentation in Fiji, image processing package.

### Example C. Identifying Synergy Between NOX5 Inhibitors and BRAF Inhibitors in Melanoma Cells

### Materials and Methods

### Cell Culture and Reagents

BRAF-mutated melanoma cells (SKMEL5, A375, WM793, SKMEL19, SKMEL28, WM164, WM88, A2058) were grown and cultured in Dulbecco's modified Eagle's medium and Ham's F-12 medium (DMEM:F12, 1:1. Cat #11330-032). Media were obtained from Gibco (Grand Island, N.Y.) and supplemented with 10% FBS, All cells were cultured in CO2, temperature-controlled (37° C.), and humidified incubators. Cells were tested for mycoplasma before use and confirmed negative. Cells were passaged 1-2× per week and maintained as exponentially growing cultures for a maximum of less than 2.0 passages. Unless otherwise indicated, cells were seeded ˜16-24 h prior to treatment to allow cells to adhere to culture plates. Reagents/drugs were prepared in complete medium immediately prior to adding to cells by replacement.

PLX4720 (Cat #S1152) and vemurafenib (Cat #S1267) were obtained from Selleckchem (Houston, Tex.) solubilized in dimethyl sulfoxide (DMSO) at a stock concentration of 10 mM. Diphenyleneiodonium chloride (DPI) (Cat #D2926) and Apocynin (Cat #498-02-2) were obtained from Sigma-Aldrich and solubilized in DMSO at a stock concentration of 10 mM for DPI and at a stock concentration of 100 mM for Apocynin. GKT137831 (Cat #HY12298) was obtained from MedChemExpress (Monmouth Junction, N.J.) and solubilized in DMSO at a stock concentration of 10 mM. All drugs were aliquoted and stored at −20° C. until use.

### Fluorescent Imaging

To facilitate automated image processing, cells were engineered to express fluorescent fusion proteins histone 2B monomeric red fluorescent protein (H2BmRFP; Addgene plasmid #18982) and geminin1-110 monomeric azami green using replication-incompetent recombinant lentiviral particles. Cells were seeded at 1000-5000 cells per well in 96-well culture imaging plates (BD Biosciences, product #353219). DMSO and PBS were used as vehicle controls, as appropriate. Images were acquired through a 10× or 20× objective with a Cellavista High End Bioimager (SynenTec Bio Services, Münster, Germany) every 6-12 hours as 3×3 or 5×5 montages. Media containing matching concentrations of drug or vehicle in each well were replaced every three days.

### Single-Cell Derived Sublines

Sixteen SKMEL5 sublines were derived from single cells by serial dilution. Briefly, cells were serially diluted to less than 1 cell per well in 96-well imaging plates and imaged to identify wells containing a single cell. Cells were expanded in complete growth medium (in the absence of BRAFi) and sequentially transferred to 48-, 24-, and 6-well plates until sufficient numbers of cells were available for cryopreservation. All sublines were tested for their sensitivity to BRAFi prior to cryopreservation.

### RNASeq and Bioinformatics Analysis

Total RNA was isolated from untreated SKMEL5 single-cell derived sublines, each in triplicate, using Trizol isolation method (Invitrogen) according to the manufacturer's instructions. RNA samples were submitted to Vanderbilt VANTAGE Core services for quality check, where mRNA enrichment and cDNA. library preparation were done with Illumina. Tru-Seq stranded mRNA sample prep kit. Sequencing was done at Paired-End 75 bp on the Illumina HiSeq 3000. Reads were aligned to the GRCh38 human reference genome using HISAT2 and gene counts were obtained using featureCounts. All downstream analyses were performed in R using the Bioconductor framework. Differential gene expression analysis was performed on genes (after low count genes were removed) using DESeq2 pipeline. Differentially expressed genes (DEGs) were selected based on a statistical cutoff of FDR <0.01 and fold change of 2. Pathway enrichment analysis was done using WebGestalt. DESeq2 rlog-values were used for visualization of gene expression levels in heat maps. Clustering was performed using the default settings of the heatmap. 2 function in the gplots R package.

RNA Isolation and Quantitative Real-Time PCR (qPCR)

Total RNA was extracted using Trizol isolation method (Invitrogen) according to the manufacturer's instructions. cDNA synthesis was performed with QuantiTect Reverse Transcription Kit (Cat #205311) from Qiagen. RT-qPCR was performed using the RPM SYBR Green Supermix from BioRad (Cat #1708880). Amplifications were performed in BioRad CFX96 Touch™ Real-Time PCR Detection System. All experiments were done at least in 3+ technical replicates. Log2 of the transcript expressions were normalized to their expressions to SKMEL5 subline SC01. HPRT was used as housekeeping gene in all the experiments. Primers used in RT-qPCR are listed in Table 1.

TABLE 1
Primers used for RT-qPCR

| Gene | Primer |
|---|---|
| NOX5 forward | GGCTCAAGTCCTACCACTGGA |
| NOX5 reverse | GAACCGTGTACCCAGCCAAT |
| PGC1α forward | TGCCCTGGATTGTTGACATGA |
| PGC1α reverse | TTTGTCAGGCTGGGGGTAGG |
| HPRT forward | TGCTCGAGATGTGATGAAGGAG |
| HPRT reverse | TGATGTAATCCAGCAGGTCAGC |

siRNA Transfection and Knockdown

For gene silencing experiments, ON-TARGETplus Human NOX5 siRNA SMARTPool (Cat #L-010195-00) was used. ON-TARGETplus Non-Targeting pool (Cat #D-001810-10-05) was used as scrambled control. Both were obtained from Dharmacon (Lafayette, Colo.) and stored at −20° C. until use. Transfection was carried out according to manufacturer's instructions using DharmaFECT1 Transfection Reagent (Cat #T-2001-02).

### Clinical Gene Expression Analysis

Gene expression of drug-naive patient samples were used from three published papers. Gene expressions were quantile-normalized before comparison. RECIST criteria reported in the papers were used as clinical outcomes, and are grouped into either Responders (partial and complete response) or Non-Responders (stable and progressive disease).

### Seahorse Metabolic Assays

For measurement of Oxygen Consumption and Extracellular Acidification Rates, cells were plated onto 96-well plates (Seahorse Biosciences, Billerica, Mass.) at a density of 25,000-40,000 cells/well before analysis on the Seahorse XFe extracellular flux analyzer. Mitochondrial Oxygen Consumption was quantified using the Mito Stress Test kit, and glycolytic rate quantified using the Glycolysis Stress Test kit, each according to manufacturer's instructions. Briefly, assay medium was unbuffered DMEM containing either 10 mM Glucose, 2 mM Glutamine, and 1 mM Sodium Pyruvate (Mito Stress Test) or none of the aforementioned (Glyco Stress Kit). No FBS was used in assay medium.

### Statistical Analysis

All statistical analyses were done in RStudio, Version 1.0.136. Two-sided Student's t-tests were used for pairwise comparisons for comparisons in gene expressions and metabolic differences.

### Identification of Molecular Determinants of Short-Term Drug Response Variability Using Single-cell-Derived Isogenic Sublines

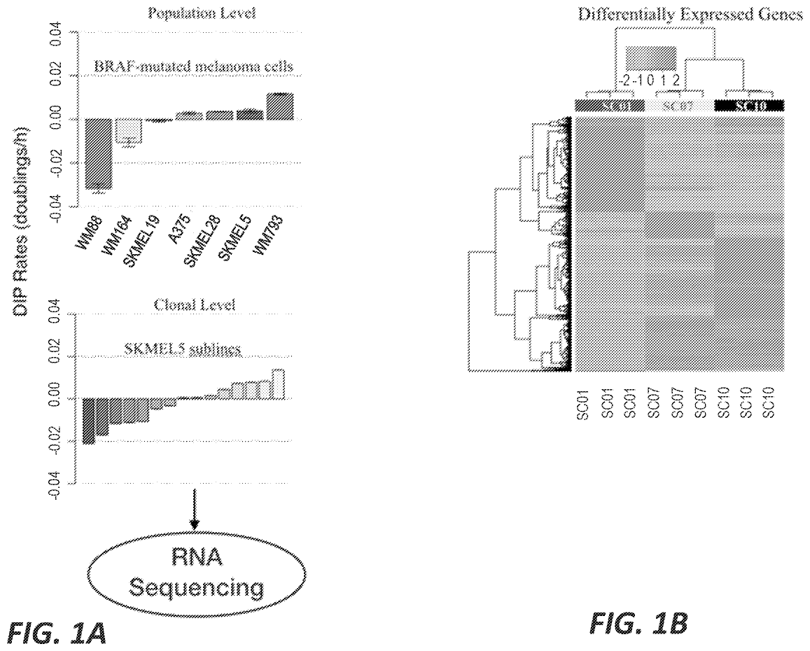

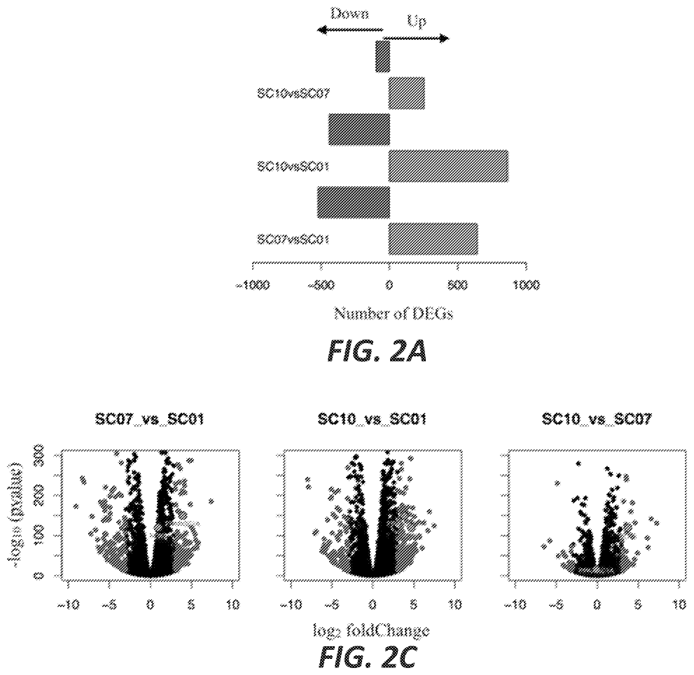

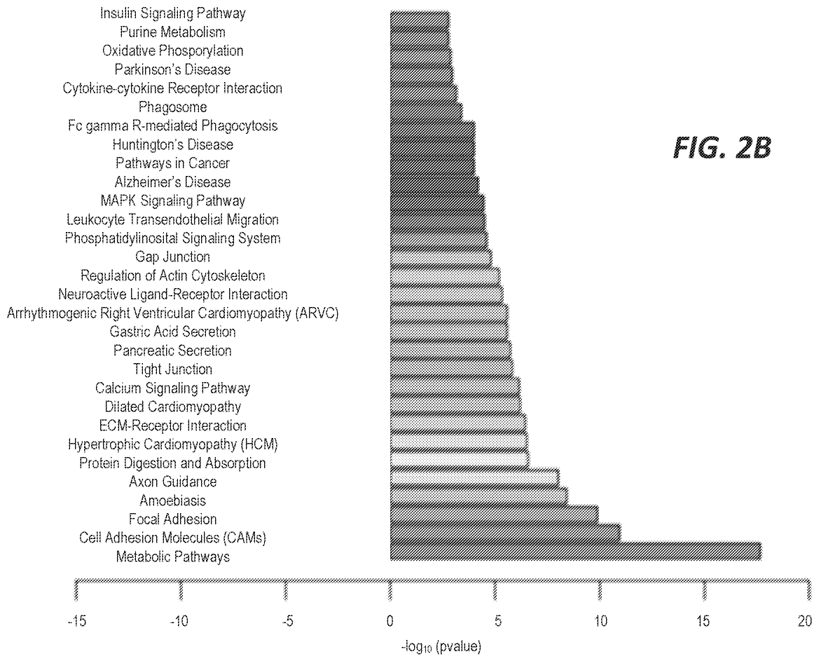

It has been reported that BRAF-mutated melanoma cell lines exhibit varying drug sensitivities to small molecule BRAFi. Briefly, it has been shown that cell lines can be ordered on a continuum based on their drug sensitivities using Drug-Induced Proliferation (DIP) rates, an unbiased metric of anti-proliferative effects of drug. Each specific melanoma cell line was studied and it was found that the short-term population level response in each cell line comprises of a wide range of clonal behaviors. Single-cell derived isogenic sublines of a BRAF-mutated melanoma cell line, SKMEL5, were used and RNASeq analysis was performed on three sublines with distinct drug responses in the short-term to probe for the molecular determinants of short-term response variability (FIG. 1A). Selected sublines had divergent drug responses in the short-term: SC01 (regressing), SC07 (stable) and SC10 (expanding). By performing pairwise comparisons between SC07 vs SC01, SC10 vs SC01, and SC10 vs SC07, differentially expressed genes (DEGs) were selected based on a statistical cutoff of FDR <0.01. Among these genes, the subset differing in a degree of enrichment by twofold or more between the sublines were focused on (FIG. 2A). The top KEGG pathways enriched in common set of upregulated DEGs included Metabolic pathways, Cell Adhesion Molecules, and Focal Adhesion (FIG. 2B).

PGC1α, a mitochondrial biogenesis transcriptional co-activator, was identified, as well as NADPH Oxidase 5 (NOX5) as potentially important determinants of sensitivities to BRAFi. While the expression of PGC1α was significantly different in SC01 compared to SC07 and SC10, its expression in SC07 and SC10 was comparable. Similarly, the expression of NOX5 was significantly different in SC10 compared to SC01 and SC07, while its expression in SC01 and SC07 was comparable (FIG. 2C). Taken together, these results suggest metabolic differences between the isogenic sublines and identify NOX5 and PGC1α as potential determinants of drug-response variability in the short-term.

### Combined Expression of PGC1α and NOX5 Correlates to BRAFi DIP Rates

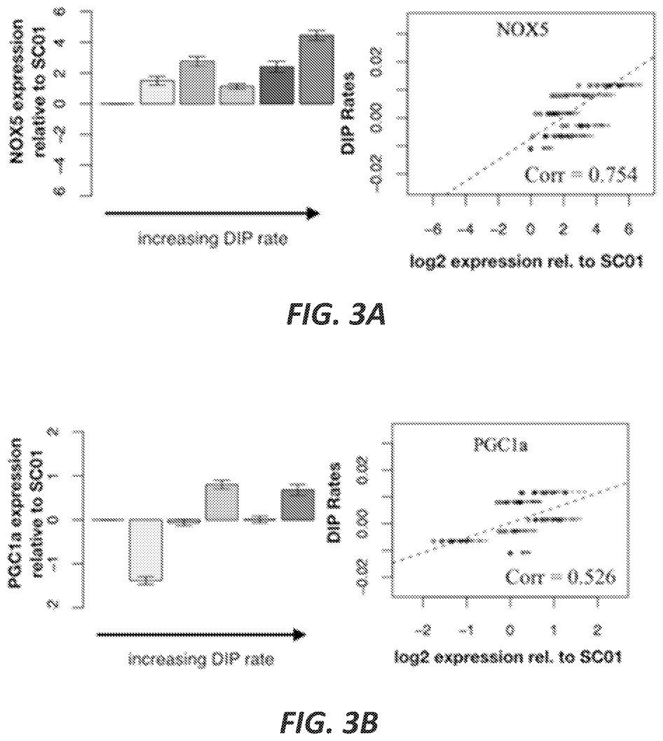

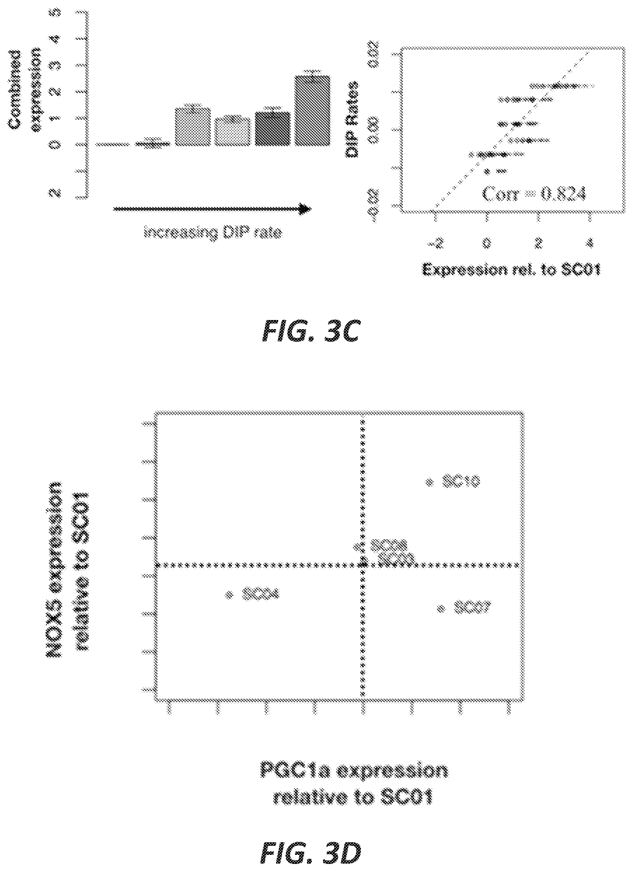

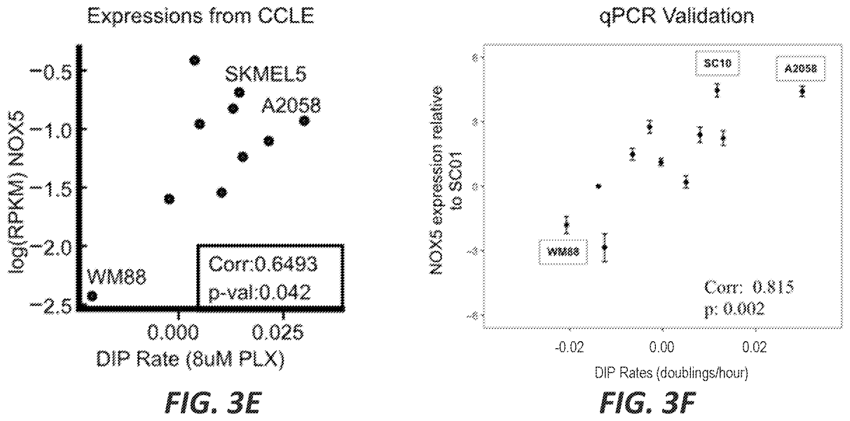

The expression of NOX5 and PGC1α was probed in more detail in six of the isogenic sublines using quantitative PCR (qPCR). Heterogeneous expression of NOX5 among clonal sublines was observed, with SC10 having the highest and SC01 with the least expression. NOX5 expression showed strong correlation (Corr: 0.754) with sensitivity of clonal sublines to BRAF-inhibition (FIG. 3A). Similarly, the expression of PGC1α was probed among six of the SKMEL5 sublines and heterogeneity was observed in its expression among sublines (FIG. 3D). In contrast to NOX5, PGC1α expression showed moderate correlation (Corr: 0.526) with the DIP rates of clonal sublines (FIG. 3B). A linear combination of NOX5 and PGC1α expression showed best correlation (Corr: 0.824) to DIP rates among sublines, much higher than expression of NOX5 (Corr: 0.754) or PGC1α (Corr: 0.526) alone (FIG. 3C). Without intending to be limited to any particular theory, these results suggest that the higher the combined expression of NOX5 and PGC1α in BRAF-mutated melanoma cell, the less sensitive the sublines are to BRAFi and vice-versa (FIG. 3C).

Additionally, examining the expression of both NOX5 and PGC1α distinguishes sublines and categorizes them based on their BRAFi sensitivity quantified in terms of DIP rates (FIG. 3D). Furthermore, to examine how the expression of these two genes (NOX5 and PGC1α) vary among BRAF-mutated melanoma cell lines, five BRAF-mutated melanoma cells were probed: sensitive (WM88 and WM164), stable (A375) and insensitive (WM115 and A2058), selected based on their BRAFi sensitivities. NOX5 but not PGC1α expression showed strong correlation (Corr: 0.92 vs Corr: 0.28) to BRAFi DIP rates. Similar to single-cell derived isogenic sublines, combined expression of NOX5 and PGC1α showed best correlation (Corr: 0.94) to BRAFi sensitivities among cell lines. Without intending to be limited to any particular theory, these results suggest that cumulative expression of NOX5 and PGC1α correlates with sensitivities of melanoma cell lines including isogenic sublines to BRAFi.

NADPH Oxidase (NOX) Inhibition Synergizes with BRAF-Inhibition in BRAF-Mutated Melanoma Cells

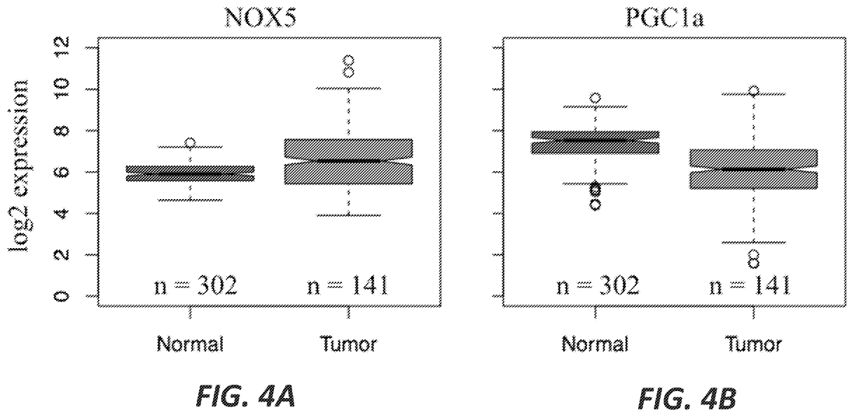

NOX5 expression is significantly higher in tumor skin tissues compared to normal tissues, whereas PGC1α expression was higher in normal tissues (FIGS. 4A and 4B). The effects of NOX inhibitors in combination with BRAFi were tested for treatment of melanoma based on these results. Three different NOX inhibitors (DPI, Apocynin and GKT137831) were selected.

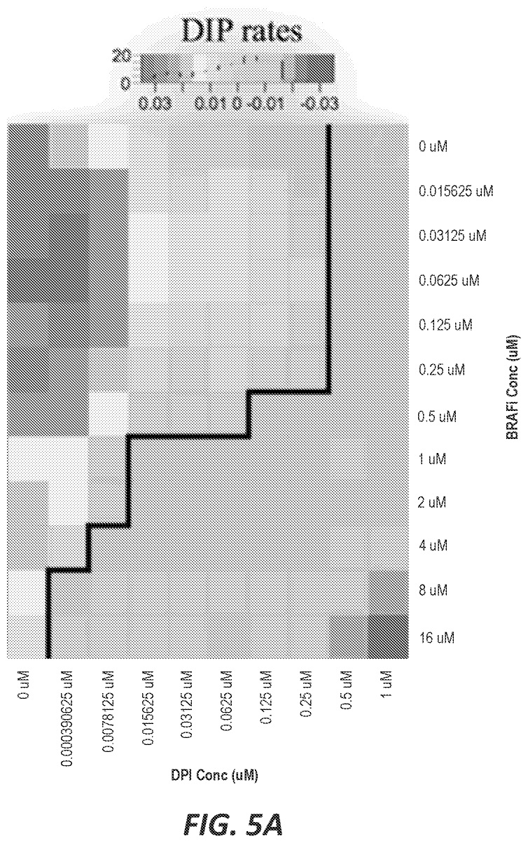

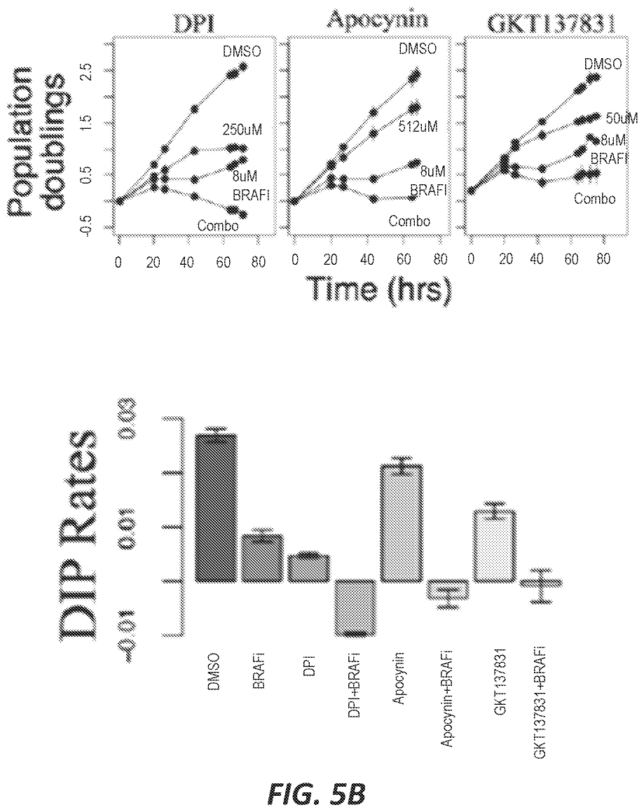

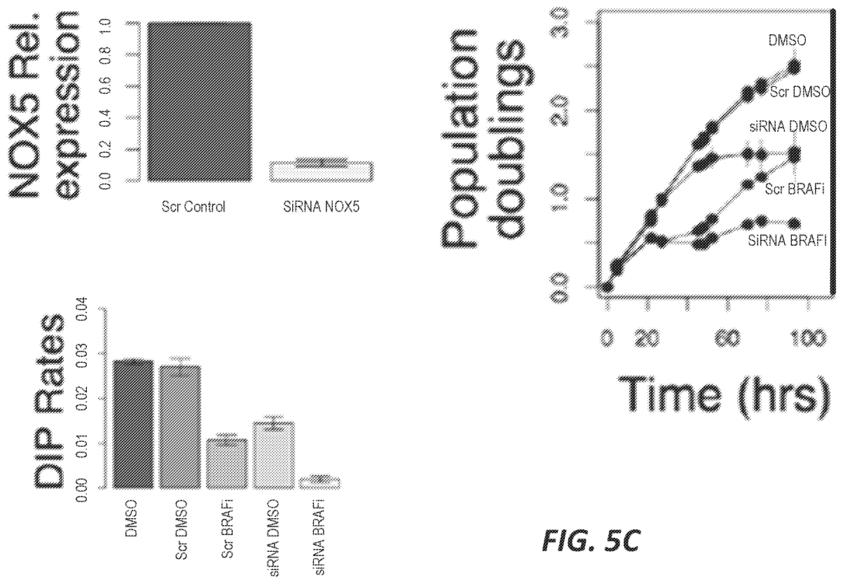

The effects of the combination in cell lines that have higher expression of NOX5 were studied, including one isogenic sublines: SC10 and A2058. These two cell lines are also cells with positive DIP rates under BRAF-inhibition. While NADPH Oxidase (NOX) inhibitors exhibited concentration-dependent anti-proliferative effects, their combination with BRAF-inhibitor, PLX4720 enhanced the effects of BRAFi. In all three NOX-inhibitors used, the NOX-inhibition synergized with BRAF-inhibition and enhanced the anti-proliferative effects of BRAF-inhibition (FIGS. 5A and 5B). Since the selected NOX-inhibitors are not NOX5 specific, NOX5 knockdown was also studied in order to determine whether it enhances the effects of BRAFi in melanoma cells.

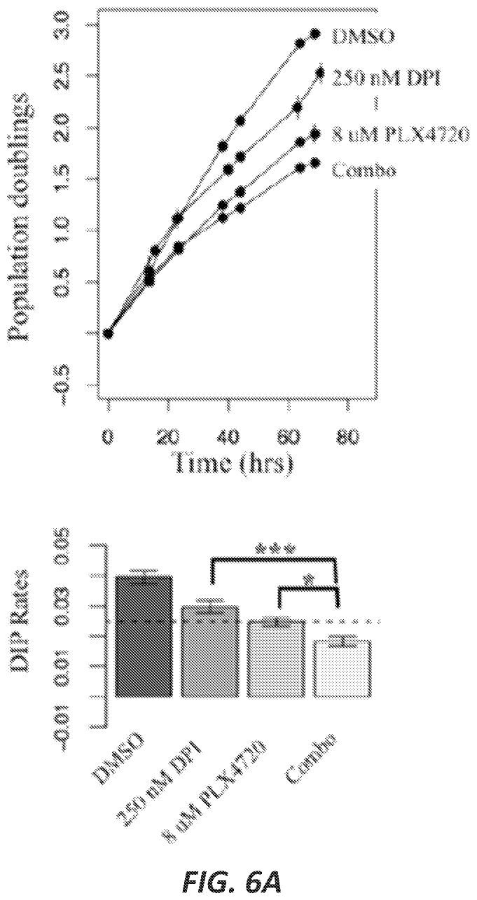

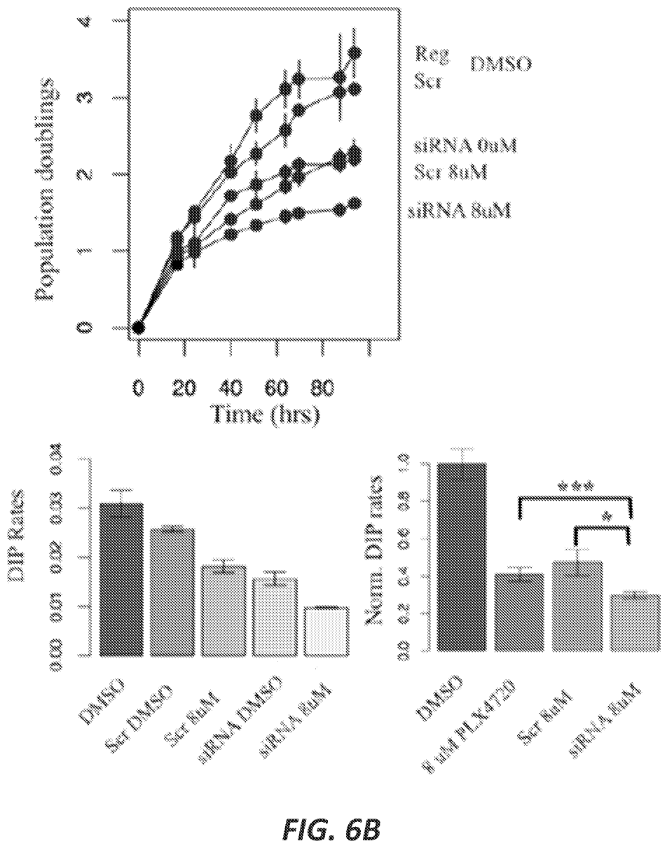

For this, siRNA-mediated approach was used to knock down NOX5 and the knockdown was validated by qPCR. More than 80% knockdown of NOX5 was observed and knockdown significantly slowed proliferation rates of cells compared to scrambled control (FIG. 5C). These results show that siRNA-mediated knockdown enhanced the effects of BRAFi (FIG. 5C). Similar results were observed for both drug combination and NOX5 knockdown in A2058 cells (FIGS. 6A and 6B). Without intending to be limited to any particular theory, these results suggest that NADPH Oxidase (NOX) inhibition, in combination with BRAFi, enhances the effects of anti-proliferative effects of BRAF-inhibition and may provide superior outcomes in treatment of BRAF-mutated melanoma.

High NOX5 Expression Correlates with Poor Clinical Outcome

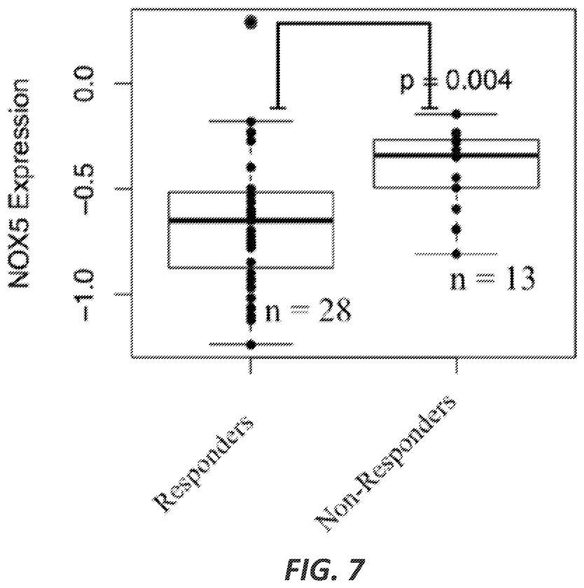

To directly investigate NOX5 expression and its correlation to patient treatment outcome, a study was conducted on three previous clinical trials for which gene expression of drug-naive patient samples and their clinical outcomes data were available. For this analysis, patients who underwent either BRAN monotherapy or BRAFi combination with MEKi were selected. Clinical outcomes were grouped into two categories: Responders (includes complete and partial response) and Non-Responders (includes stable and progressive disease) using Response Evaluation Criteria in Solid Tumors (RECIST) categories. NOX5 expression was higher in patients whose tumors under treatment were classified as Non-Responders (n=28) than in patients whose tumors responded (n=13) (t-test, p=0.004). Although there was some overlap in NOX5 expression between two groups, NOX5 expression was significantly higher in Non-Responders than in Responders (FIG. 7), suggesting the potential role of NOX5 in limiting the therapeutic efficacy of MAPK-inhibitors. Without intending to be limited to any particular theory, the analysis reveals that high NOX5 expression correlates with poor clinical outcome in BRAF-mutated melanoma patients treated with MAPK-inhibitors. In other words, these results indicate that NOX5 expression could identify BRAF-mutated melanoma patients that are less likely to respond to conventional BRAF-inhibition or combinational approaches that target MAPK pathway.

### Expressions of NOX5 and PGC1α in Melanoma Cells Correlate to Glycolytic Reserve and Mitochondrial Respiration Respectively

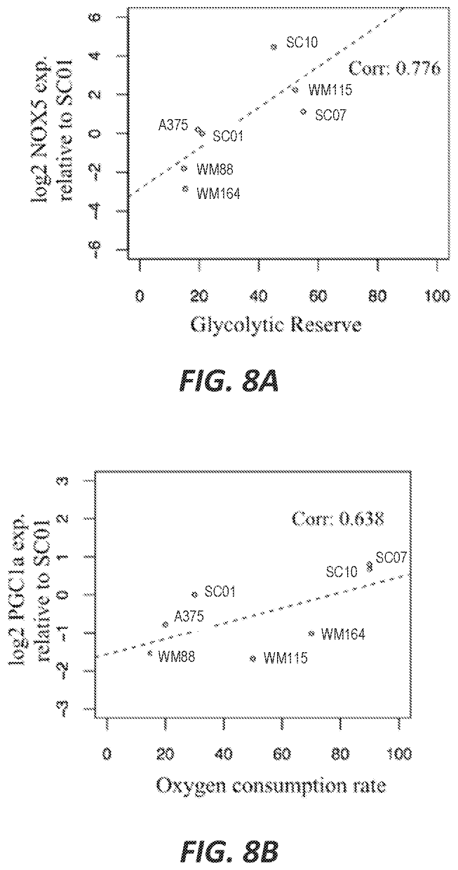

To examine the metabolic roles of NOX5 and PGC1α in melanoma cells, extracellular acidification rate (SCAR) and oxygen consumption rate (OCR) was measured in BRAF-mutated melanoma cells including isogenic sublines using Seahorse flux analyzer. From the metabolic parameters extracted from Seahorse assays, glycolytic, reserve showed a strong correlation (Corr: 0.776) with NOX5 expression in the melanoma cells (FIG. 8A). Cells with higher glycolytic reserve were mostly insensitive to BRAF-inhibition. Without intending to be limited to any particular theory, it can be postulated that higher glycolytic reserve due to an elevated NOX5 expression helps BRAF-mutated melanoma cells survive an initial BRAF-inhibition. Likewise, PGC1α expression showed moderate correlation (Corr: 0.638) with differences in mitochondrial function in melanoma cells quantified in terms of oxygen consumption rate (OCR) (FIG. 8B). Because enhanced NOX5 expression drives glucose utilization and PGC1α expression regulates mitochondrial respiration, collectively, these results suggest that BRAF-mutated melanoma cells may occupy distinct metabolic states defined by the combined expression of NOX5 and PGC1α. Without intending to be limited to any particular theory, these expressions, which define how robust different metabolic programs are in melanoma cells, could explain an initial drug response variability in BRAF-mutated melanoma cells.

The entire disclosure of each of the patent documents and scientific articles referred to herein is incorporated by reference for all purposes.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

EXAMPLE 1: A method of treating cancer in a patient, comprising administering to a patient in need thereof a therapeutically effective amount of a first therapeutic agent and a second therapeutic agent to treat the cancer, wherein the first therapeutic agent comprises a BRAF inhibitor and/or a MEK inhibitor, and the second therapeutic agent inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1 or EPHA2.

EXAMPLE 2: The method of example 1, wherein the cancer is melanoma.

EXAMPLE 3: The method of example 2, wherein the melanoma is a superficial spreading melanoma, nodular melanoma, acral-lentiginous melanoma, lentigo maligna melanoma, amelanotic melanoma, desmoplastic melanoma, ocular melanoma, nevoid melanoma, or spitzoid melanoma.

EXAMPLE 4: The method of example 1, wherein the cancer is metastatic melanoma.

EXAMPLE 5: The method of example 2, wherein the melanoma is non-metastatic.

EXAMPLE 6: The method of any one of examples 1-5, wherein the cancer has a BRAF mutation.

EXAMPLE 7: The method of any one of examples 1-6, wherein the cancer has a BRAF mutation.

EXAMPLE 8: The method of any one of examples 1-7, wherein the patient has not previously received a BRAF inhibitor for treatment of the cancer.

EXAMPLE 9: The method of any one of examples 1-8, wherein the cancer has been in remission for at least one month.

EXAMPLE 10: The method of any one of examples 1-9, wherein the cancer is characterized by elevated expression levels of the target.

EXAMPLE 11: The method of any one of examples 1-10, wherein the cancer is characterized by elevated functional activity by the target.

EXAMPLE 12: The method of any one of examples 1-11, wherein the cancer is characterized by an elevated oxidation state.

EXAMPLE 13: The method of any one of examples 1-12, wherein the first therapeutic agent comprises a small organic compound.

EXAMPLE 14: The method of any one of examples 1-14, wherein the first therapeutic agent comprises an antibody, antibody-drug conjugate, an oligonucleotide, or immunoglobulin scaffold.

EXAMPLE 15: The method of any one of examples 4, wherein the first therapeutic agent comprises a BRAF inhibitor.

EXAMPLE 16: The method of any one of examples 1-12, wherein the first therapeutic agent comprises a BRAF inhibitor selected from the group consisting of dabrafenib, Plx4720, Raf265, vemurafenib, and a pharmaceutically acceptable salt of any of the foregoing.

EXAMPLE 17: The method of any one of examples 1-16, wherein the first therapeutic agent comprises a MEK inhibitor.

EXAMPLE 18: The method of any one of examples 1-16, wherein the first therapeutic agent comprises a MEK inhibitor selected from the group consisting of binimetinib, cobimetinib, Pd98059, selumetinib, trametinib, and a pharmaceutically acceptable salt of any of the foregoing.

EXAMPLE 19: The method of any one of examples 1-18, wherein the second therapeutic agent comprises a small organic compound.

EXAMPLE 20: The method of any one of examples 1-18, wherein the second therapeutic agent comprises an antibody.

EXAMPLE 21: The method of any one of examples 1-20, wherein the second therapeutic agent inhibits NOX5.

EXAMPLE 22: The method of any one of examples 1-18, wherein the second therapeutic agent is a NOX5 inhibitor selected from the group consisting of apocynin, VAS2870, and a pharmaceutically acceptable salt of either of the foregoing.

EXAMPLE 23: The method of any one of examples 8, wherein the second therapeutic agent is diphenyleneiodonium chloride.

EXAMPLE 24: The method of any one of examples 21-23, wherein the cancer expresses elevated levels of NOX5.

EXAMPLE 25: The method of any one of examples 1-20, wherein the second therapeutic agent inhibits SLC7A11.

EXAMPLE 26: The method of any one of examples 1-18, wherein the second therapeutic agent is a SLC7A11 inhibitor selected from the group consisting of sulfasalazine or a pharmaceutically acceptable salt thereof, erastin or a pharmaceutically acceptable salt thereof, an AgilVax vaccine, and an AgilVax antibody.

EXAMPLE 27: The method of any one of examples 1-20, wherein the second therapeutic agent inhibits GSH.

EXAMPLE 28: The method of any one of examples 1-18. wherein the second therapeutic agent is a GSH inhibitor selected from the group consisting of buthionine sulfoximine and a pharmaceutically acceptable salt thereof.

EXAMPLE 29: The method of any one of examples wherein the second therapeutic agent inhibits GPX.

EXAMPLE 30: The method of any one of examples wherein the second therapeutic agent inhibits CYBA.

EXAMPLE 31: The method of any one of examples 1-20, wherein the second therapeutic agent inhibits EDG2.

EXAMPLE 32: The method of any one of examples 1-20, wherein the second therapeutic agent inhibits PPP1CC.

EXAMPLE 33: The method of any one of examples 1-20, wherein the second therapeutic agent inhibits PP1C.

EXAMPLE 34: The method of any one of examples 1-20, wherein the second therapeutic agent inhibits PP1gamma.

EXAMPLE 35: The method of any one of examples 1-20, wherein the second therapeutic agent inhibits ROCK2.

EXAMPLE 36: The method of any one of examples 1-20, wherein the second therapeutic agent inhibits RPS6KA2.

EXAMPLE 37: The method of any one of examples wherein the second therapeutic agent inhibits SYK.

EXAMPLE 38: The method of any one of examples 1-20, wherein the second therapeutic agent inhibits AKT1.

EXAMPLE 39: The method of any one of examples wherein the second therapeutic agent inhibits AKT2.

EXAMPLE 40: The method of any one of examples 1-20, wherein the second therapeutic agent inhibits BP.

EXAMPLE 41: The method of any one of examples 1-20, wherein the second therapeutic agent inhibits GSR.

EXAMPLE 42: The method of any one of examples 1-20, wherein the second therapeutic agent inhibits G6PD.

EXAMPLE 43: The method of any one of examples 1-20, wherein the second therapeutic agent inhibits ABCB5.

EXAMPLE 44: The method of any one of examples 1-20, wherein the second therapeutic agent inhibits EPHA(i).

EXAMPLE 45: The method of any one of examples 1-20, wherein the second therapeutic agent inhibits DLK1.

EXAMPLE 46: The method of any one of examples 1-20, wherein the second therapeutic agent inhibits IDH1.

EXAMPLE 47: The method of any one of examples 1-20, wherein the second therapeutic agent inhibits ME2/3.

EXAMPLE 48: The method of any one of examples 1-20, wherein the second therapeutic agent inhibits HTATIP2.

EXAMPLE 49: The method of any one of examples 1-20, wherein the second therapeutic agent inhibits DKK1.

EXAMPLE 50: The method of any one of examples 1-20, wherein the second therapeutic agent inhibits RAC3.

EXAMPLE 51: The method of any one of examples 1-20, wherein the second therapeutic agent inhibits UQCRB.

EXAMPLE 52: The method of any one of examples 1-20, wherein the second therapeutic agent inhibits ERBB4.

EXAMPLE 53: The method of any one of examples 1-20, wherein the second therapeutic agent inhibits IP3.

EXAMPLE 54: The method of any one of examples 1-20, wherein the second therapeutic agent inhibits MCU.

EXAMPLE 55: The method of any one of examples 1-20, wherein the second therapeutic agent inhibits EPHA2.

EXAMPLE 56: The method of any one of examples 1-20, wherein the second therapeutic agent inhibits SLC16A7.

EXAMPLE 57: The method of any one of examples 1-20, wherein the second therapeutic agent inhibits ELF3.

EXAMPLE 58: The method of any one of examples 1-20, wherein the second therapeutic agent inhibits NROB1.

EXAMPLE 59: The method of any one of examples 1-58, wherein there is at least a 20% reduction in the size of at least one localized cancer in the patient.

EXAMPLE 60: The method of any one of examples 1-58, wherein there is at least a 35% reduction in the size of at least one localized cancer in the patient.

EXAMPLE 61: The method of any one of examples 1-58, wherein there is at least a 50% reduction in the size of at least one localized cancer in the patient.

EXAMPLE 62: The method of any one of examples 1-61, wherein there is at least a 20% reduction in the distribution of the cancer in the patient.

EXAMPLE 63: The method of any one of examples 1-61, wherein there is at least a 35% reduction in the distribution of the cancer in the patient.

EXAMPLE 64: The method of any one of examples 1-61, Therein there is at least a 50% reduction in the distribution of the cancer in the patient.

EXAMPLE 65: The method of any one of examples 1-64, further comprising selecting the patient for treatment by analyzing a sample from the patient to identify at least one of: (a) elevated expression levels of at least one target; (b) elevated functional activity of at least one target; or (c) elevated oxidation state of the cancer.

EXAMPLE 66: A pharmaceutical composition comprising: (a) BRAF inhibitor aid/or a MEK inhibitor; (b) a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, DKK1, RAC3, UQCRB, ERBB4, IP3, MCU, SLC16A7, ELF3, NROB1 or EPHA2; and (c) a pharmaceutically acceptable carrier.

What is claimed is:

1. A method of treating cancer in a patient, comprising administering to a patient in need thereof a therapeutically effective amount of a first therapeutic agent and a second therapeutic agent to treat the cancer, wherein the first therapeutic agent comprises a BRAF inhibitor and/or MEK inhibitor, and the second therapeutic agent inhibits at least one target impacting an oxidation state of the cancer, wherein the at least one target is selected from a group consisting of: NOX5, SLC7A11, GSH, GPX, CYBA, EDG2, PPP1CC, PP1C, PP1gamma, ROCK2, RPS6KA2, SYK, AKT1, AKT2, BP, GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1, and EPHA2.

2. The method of claim 1, wherein the cancer is a melanoma selected from a group consisting of: a superficial spreading melanoma, nodular melanoma, acral-lentiginous melanoma, lentigo maligna melanoma, amelanotic melanoma, desmoplastic melanoma, ocular melanoma, nevoid melanoma, and spitzoid melanoma.

3. The method of claim 1, wherein the cancer has a BRAF mutation.

4. The method of claim 1. wherein the cancer has a KRAS mutation.

5. The method of claim 1, wherein the patient has not previously received a BRAF inhibitor for treatment of the cancer.

6. The method of claim 1, wherein the cancer has been in remission for at least one month.

7. The method of claim 1, wherein the cancer is characterized by at least one selected from a group consisting of elevated expression levels of the target, elevated functional activity by the target, and an elevated oxidation state

8. The method of claim 1, wherein the first therapeutic agent comprises a small organic compound.

9. The method of claim 1, wherein the first therapeutic agent comprises at least one selected from a group consisting of an antibody, antibody-drug conjugate, an oligonucleotide, or immunoglobulin scaffold.

10. The method of claim 1, wherein the first therapeutic agent comprises a BRAF inhibitor selected from a group consisting of dabrafenib, Plx4720, Raf265, vernurafenib, and a pharmaceutically acceptable salt of any of the foregoing.

11. The method of claim 1, wherein the first therapeutic agent comprises a MEK inhibitor selected from a group consisting of binimetinib, cobimetinib, Pd98059, selumetinib, trametinib, and a pharmaceutically acceptable salt of any of the foregoing.

12. The method of claim 1, wherein the second therapeutic agent comprises a small organic compound.

13. The method of claim 1, wherein the second therapeutic agent comprises an antibody

14. The method of claim 1, wherein the second therapeutic agent is a NOX5 inhibitor selected from a group consisting of apocynin, VAS2870, and a pharmaceutically acceptable salt of either of the foregoing.

15. The method of claim 1, wherein the second therapeutic agent is diphenyleneiodonium chloride.

16. The method of claim 1, wherein the second therapeutic agent is a SLC7A11 inhibitor selected from the group consisting of sulfasalazine or a pharmaceutically acceptable salt thereof, erastin or a pharmaceutically acceptable salt thereof, an AgilVax vaccine, and an AgilVax antibody.

17. The method of claim 1, wherein the second therapeutic agent is a GSH inhibitor selected from the group consisting of buthionine sulfoximine and a pharmaceutically acceptable salt thereof.

18. The method of claim 1, further comprising selecting the patient for treatment by analyzing a sample from the patient to identify at least one of
elevated expression levels of at least one target;
elevated functional activity of at least one target; or
elevated oxidation state of the cancer.

19. A pharmaceutical composition comprising:
a BRAF inhibitor and/or a MEK inhibitor;
a second therapeutic agent that inhibits at least one of the following targets impacting oxidation state of the cancer: NOX5, SLC7A11, GSH, GPX, CYBA, EDG2, PPP1CC, PP1C, PP1gamma, ROCK2, RPS6KA2, SYK, AKT1, AKT2, BP. GSR, G6PD, ABCB5, EPHA(i), DLK1, IDH1, ME2/3, HTATIP2, DKK1, RAC3, UQCRB, ERBB4, IP3, MCU, SLC16A7, ELF3, NROB1, or EPHA2; and
a pharmaceutically acceptable carrier.


SEQUENCE LISTING (6 sequences)

SEQ ID NO: 1 - DNA, 21 bp, Artificial Sequence
  Primer
ggctcaagtcctaccactgga

SEQ ID NO: 2 - DNA, 20 bp, Artificial Sequence
  Primer
gaaccgtgtacccagccaat

SEQ ID NO: 3 - DNA, 21 bp, Artificial Sequence
  Primer
tgccctggattgttgacatga

SEQ ID NO: 4 - DNA, 20 bp, Artificial Sequence
  Primer
tttgtcaggctgggggtagg

SEQ ID NO: 5 - DNA, 22 bp, Artificial Sequence
  Primer
tgctcgagatgtgatgaaggag

SEQ ID NO: 6 - DNA, 22 bp, Artificial Sequence
  Primer
tgatgtaatccagcaggtcagc